(19) United States
(12) Patent Application Publication
(10) Pub. No.: US 2021/0132072 A1
(43) Pub. Date: May 6, 2021

(54) PODOPLANIN POSITIVE MACROPHAGES

(71) Applicants: VIB VZW, Gent (BE); KATHOLIEKE UNIVERSITEIT LEUVEN, K.U.LEUVEN R&D, Leuven (BE)

(72) Inventors: Massimiliano Mazzone, Kessel-Lo (BE); Pawel Bieniasz-Krzywiec, Brusse (BE)

(21) Appl. No.: 16/759,431

(22) PCT Filed: Oct. 26, 2018
(86) PCT No.: PCT/EP2018/079425

§ 371 Date: Apr. 27, 2020

(30) Foreign Application Priority Data
Oct. 26, 2017 (EP) 17198684.7

(51) Int. Cl. G01N 33/574; G01N 33/50; C07K 16/30

(52) U.S. Cl. CPC G01N 33/57492; G01N 33/5055; C07K 2317/31; C07K 16/3092; G01N 33/5011

(57) ABSTRACT

The invention relates to the field of tumor metastasis and peritumoral lymphangiogenesis. More specifically, podoplanin present on a subset of tumor-associated macrophages was identified as target for treating or inhibiting tumor metastasis and peritumoral lymphangiogenesis, and podoplanin-positive macrophages were identified as biomarker for lymphatic metastasis. The invention further relates to screening methods for identifying compounds capable of neutralizing podoplanin-positive macrophages, and to methods and kits for tumor analysis or for lymph vessel analysis.

### FIELD OF THE INVENTION

The invention relates to the field of tumor metastasis and peritumoral lymphangiogenesis. More specifically, podoplanin present on a subset of tumor-associated macrophages was identified as target for treating or inhibiting tumor metastasis and peritumoral lymphangiogenesis, and podoplanin-positive macrophages were identified as biomarker for lymphatic metastasis. The invention further relates to screening methods for identifying compounds capable of neutralizing podoplanin-positive macrophages, and to methods and kits for tumor analysis or for lymph vessel analysis.

### BACKGROUND

Tumor-associated macrophages (TAMs) are an important component of the tumor stroma, both in murine models and human patients (Pollard 2004, Nat Rev Cancer 4: 71-8). TAMs can promote tumor-growth by affecting angiogenesis, immune suppression and invasion and metastasis (Lin et al. 2006, Cancer Res 66: 11238-46). At the tumor site, TAMs are confronted with different tumor microenvironments, leading to different TAM subsets with specialized functions and distinct molecular profiles, thus demonstrating the plasticity of macrophages (Laoui et al. 2011, Int. J. Dev. Biol., 55: 861-867). For example in mammary tumors, at least two distinct TAM subpopulations have been described, based on a differential expression of markers such as the macrophage mannose receptor (MMR or MHC II), differences in pro-angiogenic or immunosuppressive properties and intratumoral localization (normoxic/perivascular tumor areas versus hypoxic regions).

Expression of podoplanin (also known as Aggrus) in inflammatory macrophages was shown to activate platelets via CLEC-2, and presented as mechanism for extravascular platelet activation during clotting, wound healing and vascular inflammatory processes (Kerrigan et al 2012, J Thromb Haemost 10:484-486). Ugorski et al. 2016 (Am J Cancer Res 6:370-386) summarize knowledge on podoplanin; it is expressed in many cancers, and expression of podoplanin in cancer-associated fibroblasts is a marker of poor prognosis and is correlated with an increased incidence of metastasis to lymph nodes (Kunita et al. 2007, Am J Pathol 170:1337-1347). Chen et al. 2016 (Nat Commun 7:11302) discusses the interplay between podoplanin, galectin-8 and integrins in the process of pathological lymphangiogenesis. Several anti-podoplanin antibodies are disclosed in WO 2011/040565, WO 2012/075490, WO 2012/128082, WO 2015/053381, WO 2017/10463, and CN105754953. Kaneko et al. 2016 (Cancer Med doi: 10.1002/cam4.954) describes an anti-podoplanin antibody targeting a glycopeptide epitope.

### SUMMARY OF THE INVENTION

In one aspect the invention relates to methods for determining metastasis status of a tumor, comprising one or more of the steps of:
obtaining from a mammal having the tumor a sample comprising lymphatic vessel cells and/or lymph node cells;
determining presence or absence of podoplanin expressing macrophages (PEMs) in said sample;
determining the tumor to be metastatic when PEMs are present in said sample.
   In such methods, the lymphatic vessel cells and/or lymph node cells may be peritumoral lymphatic vessel cells and/or lymph node cells. Furthermore, the obtained sample may be processed for detection of lymph vessel cells and/or lymph node cells, and may be processed for detection of PEMs. Such processing may comprise detection of podoplanin expressed on the lymph vessel cells and/or lymph node cells and of podoplanin expressed on PEMs. Such processing may additionally or alternatively comprise detection of a macrophage marker different from podoplanin.

The invention also relates to methods for determining surface markers specific to PEMs, comprising one or more of the steps of:
obtaining a macrophage population expressing podoplanin and a macrophage population not expressing podoplanin;
reacting the obtained macrophage populations with a reagent specific to at least one surface molecule suspected to be present on macrophages of at least one population, wherein said surface molecule is different from podoplanin; and
characterizing a surface molecule for which the ratio of its levels present on PEMs over its levels present on macrophages not expressing podoplanin is higher than 1 as surface marker specific to PEMs.
   Surface molecules specific for PEMs and different from podoplanin characterized by such methods include the macrophage mannose receptor CD206, CD86, and CD204.

The invention further encompasses methods of screening for compounds neutralizing PEMs, comprising one or more of the steps of:
obtaining PEMs and lymphatic vessel cells and/or with lymph node cells;
assessing the interaction of the PEMs with lymphatic vessel cells and/or with lymph node cells in the presence or absence of at least one compound candidate for inhibiting such interaction;
selecting a compound capable of inhibiting interaction of the PEMs with the lymphatic vessel cells and/or with the lymph node cells as compound capable of neutralizing PEMs; and, optionally,
combining said selected compound with at least one pharmaceutically excipient to produce a pharmaceutical composition.

Alternative methods of screening for compounds neutralizing PEMs, are methods comprising the one or more of the steps of:
obtaining PEMs and galectin-8;
assessing the interaction of the PEMs with galectin-8 in the presence or absence of at least one compound candidate for inhibiting such interaction;
selecting a compound capable of inhibiting interaction between the PEMs and galectin-8 as compound capable of neutralizing PEMs; and, optionally,
combining said selected compound with at least one pharmaceutically excipient to produce a pharmaceutical composition.

Further alternative methods of the invention are methods of screening for compounds binding specifically to PEMs, wherein the compound is not binding to podoplanin on PEMs, and wherein the method is comprising one or more of the steps of:
obtaining a macrophage population expressing podoplanin and a macrophage population not expressing podoplanin;
reacting the macrophage populations with at least one compound; and
selecting a compound for which the ratio of its level of binding on PEMs over its levels of binding on macrophages not expressing podoplanin is higher than 1, and which is not binding to podoplanin on PEMs, as compound binding specifically to PEMs.

In any of the above methods, said PEMs or macrophages expressing podoplanin may be obtained from a tumor, a tumor environment and/or from a tumor stroma.

Another aspect of the invention relates to multi-specific antigen binding molecules specifically binding to PEMs, wherein said molecules are at least binding to podoplanin and to a second PEM-specific surface molecule. In particular, the second PEM-specific surface molecule may be chosen from the macrophage mannose receptor CD206, CD86, and CD204. Further in particular, such multi-specific antigen binding molecules may further comprise or be conjugated to a detectable label.

A further aspect of the invention relates to kits for determining metastasis status of a tumor comprising at least one agent for detection of PEMs. Such kits may further comprise at least one agent for detection of lymph vessel cells and/or lymph node cells. In such kits, the at least one agent for detection of PEMs may be one or more of the multi-specific antigen binding molecules described above. The methods for determining metastasis status of a tumor described above may also rely on detecting PEMs by means of one or more multi-specific antigen binding molecules described above.

The invention further relates to compounds neutralizing PEMs for use in treating or inhibiting lymphatic metastasis of a primary tumor or for use in treating or inhibiting tumor-induced lymphangiogenesis; such as e.g. after determination of the metastasis status of a tumor with a method for determining metastasis status of a tumor described above. In particular, the compound neutralizing PEMs may be a compound capable of neutralizing the interaction between PEMs and a lymphatic vessel, a lymphatic vessel cell, a lymph node, or a lymph node cell. Alternatively, the compound neutralizing PEMs may be an antibody binding to podoplanin, galectin-8 or integrin beta 1, or may be an antigen-binding fragment of any thereof.

In relation to all preceding methods, compounds or kits, the tumor may in particular be a breast tumor or be breast cancer.

### DESCRIPTION TO THE FIGURES

FIG. 1. Expression of podoplanin in tumor-infiltrating leukocytes (TILs) by FACS analysis. Panel A depicts the expression levels of podoplanin in the different TILs, panel B is the same as panel A indicating markers used to identify the different TIL-types denominated in panel A. MFI=mean fluorescent intensity.

FIG. 2. Expression of podoplanin by FACS analysis in tumor-associated macrophages (TAMs) and in circulating monocytes of tumor-bearing (tm-bearing) and healthy mice. MFI=mean fluorescent intensity.

FIG. 3. FACS-analysis of podoplanin-positive TAMs (PDPN+ TAMs) and podoplanin-negative TAMs (PDPN− TAMs). Panel A. Relative content of PDPN+ TAMs and PDPN− TAMs in the tumor-associated macrophage population. Panel B. Expression of macrophage marker CD206 in the indicated TAM populations. Panel C. Expression of macrophage marker CD204 in the indicated TAM populations. Panel D: Expression of macrophage marker CD86 in the indicated TAM populations. MFI=mean fluorescent intensity.

FIG. 4. Analysis of tumor growth in wild-type mice and in mice with a podoplanin-deficient hematopoietic system. Wild-type (WT) and podoplanin-knockout (Pdpn KO) bone marrow (BM) cells were used to reconstitute the immune system of lethally irradiated WT BalbC recipient mice thus generating WT→WT and Pdpn KO→WT chimeric mice.

Panel A. Schematic representation of model.

Panel B. Growth of 4T1 and EMT6.5 breast tumors over time as measured by tumor volume (mm3).

Panel C. Growth of 4T1 and EMT6.5 breast tumors over time as measured by tumor weight (gram).

FIG. 5. Analysis of tumor metastasis in wild-type mice and in mice with a podoplanin-deficient hematopoietic system. Wild-type (WT) and podoplanin-knockout (Pdpn KO) bone marrow (BM) cells were used to reconstitute the immune system of lethally irradiated WT BalbC recipient mice thus generating WT→WT and Pdpn KO→WT chimeric mice.

Panel A. Lung metastasis of 4T1 and EMT6.5 breast tumors as measured by number of metastatic lesions per lung.

Panel B. Lung metastatic index of 4T1 and EMT6.5 breast tumors.

FIG. 6. Analysis of tumor metastasis in wild-type mice and in mice with a podoplanin-deficient hematopoietic system. Wild-type (WT) and podoplanin-knockout (Pdpn KO) bone marrow (BM) cells were used to reconstitute the immune system of lethally irradiated WT BalbC recipient mice thus generating WT→WT and Pdpn KO→WT chimeric mice.

Panel A. Lung metastasis of 4T1 breast tumors as measured by number of metastatic area/lung area. px²: square pixel (a unit expressing microscopic area).

Panel B. Illustration of reduction of lung metastasis, the dark areas correspond to metastatic lesions.

FIG. 7. Analysis of tumor growth and metastasis in wild-type mice and in mice engineered with inducible deletion of podoplanin.

Panel A, left: growth of E0771 breast tumors over time as measured by tumor volume (mm3); Panel B, right: growth of E0771 breast tumors over time as measured by tumor weight (gram).

Panel B, left: lung metastasis of E0771 breast tumors as measured by number of metastatic lesions per lung; Panel B, right: lung metastatic index of E0771 breast tumors.

FIG. 8. Influence of podoplanin deletion on recruitment of TAMs to tumors.

Panel A. Recruitment of TAMs to 4T1 tumors. Left: results of histological analysis; right: results of FACS analysis.

Panel B. Recruitment of TAMs to E0771 tumor stroma, results of FACS analysis.

FIG. 9. Podoplanin-positive TAMs localize at lymphatic vessels in 4T1 tumors.

Panel A: localization of podoplanin-positive TAMs, but not podoplanin-negative TAMs attached to lymph vessels. Left pictures: staining of lymph vessels (and PDPN+ TAMs) for podoplanin; middle pictures: staining of TAMs (F4/80+); right pictures: merger of left and middle pictures, reveals presence of PDPN+ TAMs (indicated by arrows) associated with lymphatic vessels but not in areas free of lymphatic vessels.

Panel B, left: percentage of lymphatic vessel area (podoplanin-positive) taken by TAMs (F4/80-positive) in the lymphatic vessel area and outside of it, based on colocalization. Panel A, right: PDPN+ F4/80+ cells in the lymphatic vessel area and outside of it (ration number of cells over region of interest (ROI)).

FIG. 10. Podoplanin-positive TAMs localize at peritumoral lymphatic vessels.

Panel A. Left picture: staining of lymphatic vessels and PDPN+ TAMs for podoplanin; middle picture: staining of TAMs (F4/80+); right picture: merger of left and middle picture, reveals presence of PDPN+ TAMs (indicated by arrows).

Panel B. Analysis of perivascular TAMs after normalization to total podoplanin-positive area, indicating that TAMs associated with peritumoral lymphatic vessels are mainly podoplanin-positive.

FIG. 11. Podoplanin-positive TAMs interact with lymphatic endothelial cells.

Panel A. Left pictures: staining of lymphatic endothelial cells (LECs) for VEGFR3; middle picture: staining of TAMs (F4/80+); right pictures: merger of left and middle pictures, reveals presence of PDPN+ TAMs (indicated by arrows), but not of PDPN− TAMs, associated with lymphatic endothelial cells. 4T1 tumor model.

Panel B. Left: ratio of TAMs attached to LECs over free TAMs, which is significantly higher for PDPN+ TAMs compared to PDPN− TAMs (4T1 tumor model). Right: ratio of TAMs attached to LECs over free TAMs, which is significantly higher for PDPN+ TAMs compared to PDPN− TAMs (E0771 tumor model).

FIG. 12. Analysis of human breast cancer samples.

Panel A. In 12 patients with bilateral breast tumors in which one was giving rise to ipsilateral lymph node metastasis while the contralateral tumor was lymph node negative for metastasis (histopathologically determined), the presence of podoplanin-positive TAMs was assessed. Presence of PDPN+ TAMs in metastatic lymph nodes (lymph node positive; left bars of each patient) was significantly higher than in lymph nodes determined to be free of metastasis (lymph node negative: right bars of each patient).

Panel B. Left pictures: staining of lymph vessel and PDPN+ TAMs for podoplanin; middle pictures staining of TAMs (CD68-positive); right pictures: merger of left and middle pictures, indicating presence of PDPN+ TAMs in lymph node positive samples but not in lymph node negative samples.

FIG. 13. Depletion of PDPN+ TAMs reduces peritumoral lymphangiogenesis (4T1 tumor model). Panel A. Areas positive for peritumoral lymphangiogenesis in presence of PDPN+ TAMs (WT→WT) or in absence of PDPN+ TAMs (PDPN KO→WT). Lymph vessels were stained for Prox1.

Panel B. Analysis of peritumoral lymphangiogenesis by staining lymphatic vessels for Lyve1. Left: percentage of total Lyve1+ area in presence of PDPN+ TAMs (WT→WT) or in absence of PDPN+ TAMs (PDPN KO→WT); middle: number of Lyve1+ vessels in presence of PDPN+ TAMs (WT→WT) or in absence of PDPN+ TAMs (PDPN KO→WT); right: Lyve1+ area as measure of vessel size in presence of PDPN+ TAMs (WT→WT) or in absence of PDPN+ TAMs (PDPN KO→WT).

Panel C. Same as panel B but after staining lymphatic vessels for VEGFR3.

FIG. 14. Depletion of PDPN+ TAMs reduces peritumoral lymphangiogenesis (E0771 tumor model).

Panel A. Areas positive for peritumoral lymphangiogenesis in presence of PDPN+ TAMs (Csf1R;Pdpn^{wt/wt}) or in absence of PDPN+ TAMs (Csf1R;Pdpn^{lox/lox}). Lymph vessels were stained for Prox1.

Panel B. Same as panel A but after staining lymphatic vessels for VEGFR3.

Panel C. Impact of depletion of PDPN+ TAMs on Evans Blue dye drainage from 4T1 tumors to inguinal lymph nodes.

FIG. 15. Depletion of PDPN+ TAMs does not affect pathological corneal angiogenesis and lymphangiogenesis induced by corneal cauterization.

Panel A. Pathological corneal angiogenesis in presence of PDPN+ TAMs (WT→WT) or in absence of PDPN+ TAMs (PDPN KO→WT) as measured after CD31-staining.

Panel B. Pathological corneal lymphangiogenesis in presence of PDPN+ TAMs (WT→WT) or in absence of PDPN+ TAMs (PDPN KO→WT) as determined by counting the number of lymphatic branching points (left) or as measured after Lyve1-staining (right).

Panel C. Corneal flat mounts stained with CD31 and Lyve1; the brighter signals at the right of each picture are Lyve1+, the signals on the left correspond to CD31.

FIG. 16. Role of galectin-8.

Panel A. Left picture: staining of lymphatic vessels for podoplanin; middle picture: staining of lymphatic vessels for galectin-8; right picture: merger of left and middle picture, showing co-localization of podoplanin and galectin-8.

Panel B. Migration of bone marrow-derived macrophages (BMDM) towards soluble galectin-8 is depending on the presence of podoplanin on macrophages.

Panel C. Efficacy of Gal-8 expression silencing in cultured lymphatic endothelial cells (LECs) by siRNA. Panel D. Migration of PDPN+ BMDMs is depending on Gal-8, but not on CCL2 or CCL21. Migration of PDPN− BMDMs to CCL2 or CCL21 is not affected. LECs scrambled: lymphatic endothelial cells transfected with a negative control for siRNA (nonsense sequence), producing Gal-8 normally as opposed to LECs transfected with siGal-8.

FIG. 17. Role of galectin-8.

Panel A. Migration of BMDMs to LECs is inhibited by thiodigalactoside (TDG). HMVECs: human microvascular endothelial cells (a standard in vitro model of lymphatic endothelium).

Panel B. Migration of 4T1 TAMs towards soluble galectin-8 is dependent on podoplanin present on the TAMs, this in contrast to migration towards CCL21.

FIG. 18. Interaction of BMDMs with LEC sprouts formed on Matrigel.

Panel A. Attachment of BMDMs to LEC sprouts is dependent on podoplanin on the BMDMs.

Panel B. Length of preformed LEC sprouts is not affected by podoplanin deletion.

FIG. 19. Interaction of BMDMs with LEC sprouts formed on Matrigel further depends on integrin B1 (CD29).

Panel A. Attachment of BMDMs to LEC sprouts is dependent on podoplanin on the BMDMs. Attachment of PDPN+ BMDMs to LEC sprouts is blocked by inhibiting integrin B1.

Panel B. Length of preformed LEC sprouts is not affected by podoplanin deletion or inhibition of integrin B1.

Panel C. Transwell migration assay towards soluble galectin-8, and 20% FBS, in the presence or absence of an inhibitor of integrin B1.

FIG. 20. PDPN+ BMDMs positively affect sprouting of LECs upon co-culture on Matrigel.

FIG. 21. Analysis of tumor growth, tumor metastasis, and peritumoral lymphangiogenesis in the presence or absence of TDG in wild-type mice and in mice with a podoplanin-deficient hematopoietic system. Wild-type (WT) and podoplanin-knockout (Pdpn KO) bone marrow (BM) cells were used to reconstitute the immune system of lethally irradiated WT BalbC recipient mice thus generating WT→WT and Pdpn KO→WT chimeric mice.

Panel A. Growth of 4T1 breast tumors over time as measured by tumor volume (mm3).

Panel B. Lung metastasis of 4T1 breast tumors as measured by number of metastatic lesions per lung (left), and lung metastatic index of 4T1 breast tumors (right).

Panel C. Analysis of peritumoral lymphangiogenesis by staining lymphatic vessels for Lyve1 (left) or for VEGFR3 (right).

### DETAILED DESCRIPTION TO THE INVENTION

In work leading to this invention, it was shown that podoplanin (hereinafter also referred to as Pdpn, pdpn or PDPN) is required for the migration and adhesion of tumor-associated macrophages (TAMs) to lymphatic endothelial cells (LECs). Mechanistically, Pdpn allows clustering of integrin beta1 on macrophages, supporting the binding of this complex to galectin-8 (also referred to as gal-8, gal8, Gal-8, Gal8 or Galectin-8) that is mainly expressed by LECs. Genetic knockout of podoplanin, pharmacologic inhibition of Gal8, or a neutralizing antibody against integrin beta 1 is sufficient to inhibit macrophage migration and adhesion to LECs. In mice, macrophage-specific deletion of Pdpn does not affect total TAM infiltration but rather prevents their localization around the lymphatics in several breast cancer models. As a consequence, lymphatic vessels display a functional and density deficit while blood vessels are not affected. Reduced lymph vessel number and functionality strongly prevents breast cancer metastasis while, importantly and unexpectedly, lymph vessels in other organs are not affected/do not change. Intratumoral injection of a Gal8 inhibitor mimicked this phenotype in wild-type mice but did not display any effect when TAMs were deficient for Pdpn. In breast cancer patients, pdpn-expressing macrophage (PEM) association to lymphatics or lymph vessel-born Gal8 expression strongly correlates with the incidence of lymph node metastasis. These findings highlight the functional role of pdpn in pdpn-expressing macrophages and open new possibilities to specifically target tumor metastasis, tumor-induced lymphangiogenesis or to use pdpn-expressing macrophages as a prognostic biomarker in breast cancer.

Based hereon, the invention is defined in the following aspects and embodiments, and described in more detail hereafter. Podoplanin expressing macrophages will be interchangeably referred to hereinafter sometimes as PEMs, or as podoplanin-positive macrophages.

In one aspect, the invention relates to methods and kits for determining the metastasis status of a tumor. In essence, such methods are methods of tumor analysis, or, alternatively, methods of lymphatic vessel or lymph node analysis and may comprise one or more of the steps of:
obtaining from a mammal having the tumor a sample comprising lymphatic vessel cells and/or lymph node cells;
determining presence or absence of podoplanin expressing macrophages (PEMs) in said sample;
determining the tumor to be metastatic when PEMs are present in said sample.
   Alternatively, such methods are methods of tumor analysis, or in particular methods of lymphatic vessel and/or lymph node analysis and may comprise one or more of the steps of:
obtaining from a mammal having the tumor a sample comprising lymphatic vessel and/or lymph node cells;
processing the obtained sample for detection of lymph vessel and/or lymph node cells and for detection of podoplanin-positive macrophages;
determining presence or absence of podoplanin-positive macrophages associated with lymph vessel and/or lymph node cells in said sample;
determining the tumor to be metastatic when PEMs are present in said sample.
   In the above methods, said lymphatic vessel cells and/or lymph node cells may be peritumoral lymphatic vessel cells and/or peritumoral lymph node cells.

In such methods, the sample may be processed for detection of podoplanin in lymph vessel and/or in lymph node cells, and/or for detection of PEMs/podoplanin-positive macrophages. The sample may for instance by processed for detection of podoplanin expressed on the lymph vessel cells and/or lymph node cells and for detection of podoplanin expressed on macrophages/PEMs. The sample may also be processed for detection of macrophages (independent of the presence of podoplanin).

Such methods, may comprise (computational) merger of the podoplanin detection signal (in lymph vessel and/or lymph node cells; and in macrophages) and a macrophage detection signal (different from podoplanin detection signal) and/or may comprise detection of overlap between the podoplanin detection signal and the macrophage detection signal. In case of detection of such overlap (podoplanin signal+macrophage signal), it can be concluded that PEMs are present.

In the methods of tumor analysis, or in the methods of lymphatic vessel and/or lymph node analysis, lymph vessel and/or lymph node cells, podoplanin-positive macrophages/PEMs can be detected immunologically and/or by immunohistochemistry. The lymph vessel and/or lymph node cells can in particular be lymphatic endothelial cells; or alternatively the lymph node cells can be sentinel lymph node cells.

The invention further relates to methods for determining surface markers (other than podoplanin) present on and specific for podoplanin-positive macrophages/PEMs, wherein such methods for determining surface markers specific to PEMs are comprising one or more of the steps of:
obtaining a macrophage population expressing podoplanin and a macrophage population not expressing podoplanin;
reacting the obtained macrophage populations with a reagent specific to at least one surface molecule suspected to be present on macrophages of at least one population, wherein said surface molecule is different from podoplanin; and
characterizing a surface molecule for which the ratio of its levels present on PEMs over its levels present on macrophages not expressing podoplanin is higher than 1 as surface marker specific to PEMs.
   Alternatively, such methods for determining surface markers specific to PEMs may comprise one or more of the steps of:
obtaining podoplanin-positive and podoplanin-negative macrophage populations from a tumor, a tumor environment and/or from a tumor stroma;
reacting the obtained macrophage populations with a reagent specific to at least one surface molecule suspected to be present on macrophages of at least one population; and
identification of a surface molecule for which the ratio of its levels present on podoplanin-positive macrophages over its levels present on podoplanin-negative macrophages is higher than 1.
   In particular, such methods may be based on flow cytometry or on a microfluidic chip. Furthermore, the ratio mentioned may be higher than 2, may be around 3, or may be higher than about 3, higher than about 4, or higher than about 5. It is clear that surface markers identified via such method finds immediate utility in designing highly specific compounds binding to PEMS, such as diagnostic compounds and compounds capable of neutralizing podoplanin-positive macrophage/PEMs. Further in particular, the detected surface molecule specific for PEMs and different from podoplanin is the macrophage mannose receptor CD206, is CD86, or is CD204.

Further aspects of the invention relate to different types of screening assays.

As such, the invention includes methods of screening for compounds neutralizing PEMs, comprising the steps of:
obtaining PEMs and lymphatic vessel cells and/or with lymph node cells;
assessing the interaction of the PEMs with lymphatic vessel cells and/or with lymph node cells in the presence or absence of at least one compound candidate for inhibiting such interaction;
selecting a compound capable of inhibiting interaction of the PEMs with the lymphatic vessel cells and/or with the lymph node cells as compound capable of neutralizing PEMs; and, optionally,
combining said selected compound with at least one pharmaceutically excipient to produce a pharmaceutical composition.
   Alternatively, methods of screening for podoplanin-positive macrophage-neutralizing compounds, are methods comprising one or more of the steps of:
isolating podoplanin-positive macrophages from a tumor, a tumor environment and/or from a tumor stroma;
assessing the interaction of the isolated podoplanin-positive macrophages with lymphatic vessel cells in the presence or absence of a single compound, or in the presence or absence of single or grouped compounds being member(s) of a compound library, wherein the single compound is candidate for inhibiting such interaction or wherein the grouped compounds comprise a candidate for inhibiting such interaction; and
selecting a compound capable of inhibiting interaction between the podoplanin-positive macrophages and the lymphatic vessel cells as compound capable of neutralizing PEMS; and, optionally,
combining said selected compound with at least one pharmaceutically excipient to produce a pharmaceutical composition.
   In particular, relating to the above methods, they can be modified such as by performing the step of assessing the interaction between the PEMs and the lymphatic vessel/lymph node cells in the presence of (soluble) galectin-8. PEMs may be obtained from a tumor, a tumor environment and/or from a tumor stroma; alternatively macrophages are manipulated to express podoplanin. The tumor material, whether or not including tumor environmental material and/or tumor stroma, can be obtained e.g. upon biopsy or upon surgical resection of the tumor. The lymphatic vessel cells and/or lymph node cells may be peritumoral lymphatic vessel cells and/or peritumoral lymph node cells.

The invention also relates to methods of screening for compounds neutralizing PEMs, wherein such methods are comprising one or more of the steps of:
obtaining PEMs and galectin-8 or integrin beta 1;
assessing the interaction of the PEMs with galectin-8 or integrin beta 1 in the presence or absence of at least one compound candidate for inhibiting such interaction;
selecting a compound capable of inhibiting interaction between the PEMs and galectin-8 or integrin beta 1 as compound capable of neutralizing PEMs; and, optionally,
combining said selected compound with at least one pharmaceutically excipient to produce a pharmaceutical composition.
   Alternatively, such methods of screening for podoplanin-positive macrophage-neutralizing compounds, are methods comprising one or more of the steps of:
isolating podoplanin-positive macrophages from a tumor, a tumor environment, and/or from a tumor stroma;
assessing the interaction of isolated podoplanin-positive macrophages with galectin-8 or integrin beta 1 in the presence or absence of a single compound, or in the presence or absence of single or grouped compounds being member(s) of a compound library, wherein the single compound is candidate for inhibiting such interaction or wherein the grouped compounds comprise a candidate for inhibiting such interaction; and
selecting a compound capable of inhibiting interaction between the podoplanin-positive macrophages and galectin-8 or integrin beta 1 as compound capable of neutralizing PEMs; and, optionally,
combining said selected compound with at least one pharmaceutically excipient to produce a pharmaceutical composition.
   In particular, relating to the above methods, the assessment of the interaction of PEMs with galectin-8 or integrin beta 1 can for instance be performed by means of a cell migration assay. PEMs may be obtained from a tumor, a tumor environment and/or from a tumor stroma; alternatively macrophages are manipulated to express podoplanin.

Further methods of the invention are methods of screening for compounds binding specifically to PEMs, wherein the compound is not binding to podoplanin on PEMs, and wherein the method is comprising one or more of the steps of:
obtaining a macrophage population expressing podoplanin and a macrophage population not expressing podoplanin;
reacting the macrophage populations with at least one compound; and
selecting a compound for which the ratio of its level of binding on PEMs over its levels of binding on macrophages not expressing podoplanin is higher than 1, and which is not binding to podoplanin on PEMs, as compound binding specifically to PEMs.

Alternatively, such methods of screening for compounds binding specifically to podoplanin-positive macrophages, are methods comprising one or more of the steps of:
obtaining podoplanin-positive and podoplanin-negative macrophage populations from a tumor, a tumor environment and/or from a tumor stroma;
reacting the obtained macrophage populations with a compound or a set of compounds; and
selecting a compound for which the ratio of its level of binding on podoplanin-positive macrophages over its level of binding on podoplanin-negative macrophages is higher than 1, and which is not binding to podoplanin on podoplanin-positive macrophages, as compound binding specifically to PEMs.

In these methods the compounds may for instance be displayed by a phage library. In particular, the ratio mentioned in of such methods is higher than 1 (e.g. any number between 1 and 100-fold higher, e.g. 1.1, 1.2, 1.5, 1.7, 1.8, 2.0, 2.2, 2.5, 2.7, 3.0 fold higher, e.g. between 3 and 50-fold higher, or e.g. between 3 and 100-fold higher), or can e.g. be higher than about 2, higher than about 3, higher than about 4, or higher than about 5, or can be around 3, around 4, or around 5.

Compounds tested in the above screening methods are not limited to a specific type of the compound. In one embodiment, compound libraries (comprising at least two different compounds) are screened. Compound libraries are a large collection of stored compounds utilized for high throughput screening. Compounds in a compound library can have no relation to one another, or alternatively have a common characteristic. For example, a hypothetical compound library may contain all known compounds known to bind to a specific binding region. As would be understood by one skilled in the art, the methods of the invention are not limited to the types of compound libraries screened. For high-throughput screening, compound libraries may be used. Examples include, but are not limited to, natural compound libraries, allosteric compound libraries, peptide libraries, antibody fragment libraries, synthetic compound libraries, combinatorial chemical libraries etc. In one embodiment, high throughput screening methods involve providing a library containing a large number of compounds (candidate compounds) potentially having the desired activity. Such libraries are then screened in one or more assays, as described herein, to identify those library members that display the desired characteristic activity. The compounds thus identified can serve as conventional “hit or lead compounds” or can themselves be used as potential or actual therapeutics.

In a further aspect, the invention relates to multi-specific antigen binding molecules specifically binding to PEMs, wherein said molecules are at least binding to podoplanin and to a second PEM-specific surface molecule. In particular, the second PEM-specific surface molecule in such multi-specific antigen binding molecules may be chosen from the macrophage mannose receptor CD206, CD86, and CD204. Furthermore, such multi-specific antigen binding molecules may further comprising a detectable label or detectable moiety.

In any of the methods for determining the metastasis status of a tumor as described hereinabove, the detection of PEMs can for instance rely on the use of, or be determined by means of any of such multi-specific antigen binding molecules specifically binding to PEMs.

In a particular aspect, such multi-specific antigen binding molecules specifically binding to PEMs are compounds neutralizing PEMs or are podoplanin-positive macrophage-neutralizing compounds. Such compounds find use in (methods of) treating or inhibiting lymphatic metastasis of a primary tumor (in a mammal), or use in (methods of) treating or inhibiting tumor-induced lymphangiogenesis. Such compounds may also find use in (methods of) treating or inhibiting lymphatic metastasis of a primary tumor (in a mammal), whereby or wherein the metastasis status of a tumor is determined/confirmed with a method for determining the metastasis status of a tumor as described hereinabove. Such methods may comprise administering a (therapeutically effective amount of) a compound neutralizing PEMs or may comprise administering a (therapeutically effective amount of) a podoplanin-positive macrophage-neutralizing compound (to a mammal in need thereof), therewith treating or inhibiting lymphatic metastasis of the primary tumor, or therewith treating or inhibiting tumor-induced lymphangiogenesis. Herein, the primary tumor can in particular be breast cancer or breast tumor; the metastasis can in particular be lung metastasis; the tumor-induced lymphangiogenesis can in particular be peritumoral lymphangiogenesis; the administration can be intratumoral, peritumoral or systemic.

Compounds neutralizing PEMs or are podoplanin-positive macrophage-neutralizing compounds for the medical uses described above are not limited to the multi-specific antigen binding molecules specifically binding to PEMs described hereinabove. In general, such compounds neutralizing PEMs or podoplanin-positive macrophage-neutralizing compound may for instance be a compounds capable of neutralizing interaction, association, or binding between podoplanin on podoplanin-positive macrophages on the one hand and a lymphatic vessel, a lymphatic vessel cell, a lymph node, or a lymph node cell on the other hand. The compound neutralizing PEMs or podoplanin-positive macrophage-neutralizing compound may alternatively be a compound capable of neutralizing interaction between podoplanin on podoplanin-positive macrophages and galectin-8 or capable of neutralizing interaction between podoplanin on podoplanin-positive macrophages and integrin beta 1 (present on pdpn+ macrophages). In particular, the compound neutralizing PEMs or podoplanin-positive macrophage-neutralizing compound may be a compound capable of binding to podoplanin on podoplanin-positive macrophages and to at least one second molecule different from podoplanin and present on the podoplanin-positive macrophage. Such second molecule may for instance be the macrophage mannose receptor CD206, CD86, or CD204.

The compound neutralizing PEMs or podoplanin-positive macrophage-neutralizing compound may comprise a nanoparticle (of whatever nature) or virus-like particle, e.g. in (covalently or non-covalently) conjugated form. Such nanoparticle or virus-like particle may comprise a therapeutic agent (e.g. a known anti-cancer agent, a cytotoxic molecule, an immunomodulatory agent), a macrophage-inhibiting agent or a macrophage re-educating agent. The compound neutralizing PEMs or podoplanin-positive macrophage-neutralizing compound may comprise, or further comprise, an albumin-binding moiety, a mannose- or a galactosyl-comprising moiety, a mannosylated or galactosylated carrier (e.g. dextran), M2pep, LyP-1, or a cathepsin target sequence.

In a further aspect, the invention relates to any kit for (use in a method of) tumor analysis, such as any kit for determining metastasis status of a tumor, or for (use in a method of) analysis of lymphatic vessel and/or lymph node outlined above. Such kits in particular may be diagnostic kits. In particular, such kit is comprising one or both of an agent for detection of lymph vessel and/or lymph node cells, and an agent for detection of PEMs. Such kit may comprise for instance an agent for detection of podoplanin. Alternatively, such kit is comprising one or both of an agent for detection of podoplanin, and an agent (different from one for detection of podoplanin) for detection of macrophages. Further alternatively, such kit is comprising one or more of an agent for detection of podoplanin, an agent (different from one for detection of podoplanin) for detection of lymph vessel and/or lymph node cells, and an agent (different from one for detection of podoplanin) for detection of macrophages. In particular, such kit comprise at least as agent a multi-specific antigen binding molecule specifically binding to PEMs as described hereinabove. In particular, such kit is a kit for determining metastasis status of a tumor. The agents can, as part of the kit, be packaged separately or in any combination in one or more packages. The kit may further comprise reagents for detection of the detectable label and usually further comprises written instructions explaining its purpose and how to use it.

The invention further relates to compounds binding to podoplanin, galectin 8 and/or integrin beta 1 (in particular such compounds are, or are including antibodies in whatever format, or are or are including antigen-binding fragments thereof) for use in treating or inhibiting tumor metastasis, in particular lymphatic tumor metastasis, and/or for use in treating of inhibiting tumor-induced lymphangiogenesis, in particular peritumoral lymphangiogenesis. Alternatively, the invention relates to methods for treating or inhibiting tumor metastasis, in particular lymphatic tumor metastasis, and/or for treating of inhibiting tumor-induced lymphangiogenesis, in particular peritumoral lymphangiogenesis, in a mammal, comprising administration of a (therapeutically effective amount of) an anti-podoplanin antibody, anti-galectin 8 antibody and/or anti-integrin beta 1 antibody to the mammal (in need thereof), therewith treating or inhibiting tumor metastasis, in particular lymphatic tumor metastasis, and/or treating of inhibiting tumor-induced lymphangiogenesis. In particular, the tumor is e.g. breast cancer, and the metastasis is e.g. lung metastasis. Amino acid sequences of human podoplanin, galectin 8 and integrin beta 1 are available in GenBank.

Before describing further aspects and embodiments of the invention some items already mentioned above are explained in more detail.

### Tumor, Cancer, Neoplasm

The terms tumor and cancer are sometimes used interchangeably but can be distinguished from each other. A tumor refers to “a mass” which can be benign (more or less harmless) or malignant (cancerous). A cancer is a threatening type of tumor. A tumor is sometimes referred to as a neoplasm: an abnormal cell growth, usually faster compared to growth of normal cells. Benign tumors or neoplasms are nonmalignant/non-cancerous, are usually localized and usually do not spread/metastasize to other locations. Because of their size, they can affect neighboring organs and may therefore need removal and/or treatment. A cancer, malignant tumor or malignant neoplasm is cancerous in nature, can metastasize, and sometimes re-occurs at the site from which it was removed (relapse).

The initial site where a cancer starts to develop gives rise to the primary cancer. When cancer cells break away from the primary cancer (“seed”), they can move (via blood or lymph fluid) to another site even remote from the initial site. If the other site allows settlement and growth of these moving cancer cells, a new cancer, called secondary cancer, can emerge (“soil”). The process leading to secondary cancer is also termed metastasis, and secondary cancers are also termed metastases. For instance, liver cancer can arise as primary cancer, but can also be a secondary cancer originating from a primary breast cancer, bowel cancer or lung cancer; some types of cancer show an organ-specific pattern of metastasis.

Most cancer deaths are in fact caused by metastases, rather than by primary tumors (Chambers et al. 2002, Nature Rev Cancer 2:563-572).

### Lymph Node—Sentinel Lymph Node

Sentinel lymph node (SLN) biopsy is a common procedure for breast (and some other) cancer patients. The sentinel lymph node is the hypothetical first lymph node or group of nodes draining a cancer and thus the first destination of disseminating/metastatic tumor or cancer cells. Sentinel lymph node biopsy or sentinel node procedure is instrumental in determining metastasis/prognosis and provides guidance for (post-operative) therapy. An advantage of the sentinel node procedure is that it decreases the number of unnecessary lymph node dissections, thereby reducing the risk of lymphedema. Kurosumi et al. 2007 (Breast Cancer 14:342-349) reviewed the procedure and the recommendations made by the Philadelphia consensus meeting held in April 2001 in this respect: sections less than 2.0 mm in thickness should be prepared and examined to detect metastasis larger than 2.0 mm. Hematoxylin-eosin (HE) staining is often applied. In addition, immunohistochemistry for cytokeratin is considered to be useful to detect isolated tumor cells (ITC) in the representative sections. On the other hand, real-time reverse transcriptase-polymerase chain reaction (RT-PCR) might detect micrometastasis (larger than 0.2 mm) and expected to be used as a routine method instead of histopathological examination. Visual identification of sentinel lymph nodes is achieved e.g. by the injection of dyes that bind strongly to endogenous albumin, targeting the dyes to the lymph nodes.

Harisinghani et al. 2003 (N Engl J Med 348:2491-2499) reported on the use of high-resolution MRI with magnetic nanoparticles allowing the detection of small and otherwise undetectable lymph-node metastases in patients with prostate cancer.

### Targeting Podoplanin-Positive Macrophages

Specific delivery of a drug to tumor-associated macrophages, or other cell, largely depends on the availability of a specific target(s) molecule on the targeted cell and/or on the availability of a means to restrict drug action to a specific target (e.g. limited activation of a drug at and/or near the target). Obviously, podoplanin is a first target of choice present on the podoplanin-positive macrophages identified herein. Specificity of compounds intended to neutralize podoplanin-positive macrophages can be increased by including a second target specific to podoplanin-positive macrophages. Surface markers other than podoplanin and enriched in podoplanin-positive macrophages compared to/versus podoplanin-negative macrophages include CD86, CD204, and CD206 (also known as macrophage mannose receptor or pattern-recognition receptor).

The intent of neutralization of podoplanin-positive macrophages can be to prevent their interaction with or binding to lymphatic vessel cells and/or lymph node cells (such as sentinel lymph node cells) and/or lymphatic endothelial cells. It can also be to inhibit, stall or suppress podoplanin-positive macrophage function such as by re-programming, re-orienting, re-educating, or re-polarizing podoplanin-positive macrophages such as by inducing the “M1” phenotype. Neutralizing podoplanin-positive macrophages can also include the elimination, killing, ablation or depletion of these cells, e.g. by means of a cytotoxic agent or an apoptosis-inducing agent. As summarized by Mantovani et al. 2017 (Nat Rev Clin Oncol 14:399-416), several general strategies targeting macrophages in anticancer therapy are being explored such as aiming at inhibiting the localization of these cells at tumour sites (e.g. by interfering with chemokines and/or chemokine receptors; in the current instance galectin-8 may be considered as kind of chemokine, and podoplanin localized on the macrophage as kind of chemokine receptor), aiming at inhibiting functions of macrophages related to the promotion of tumour progression (which also may involve e.g. chemokines/chemokine receptors, e.g. CCL5-CCR5 axis; or which may involve re-education of tumor promoting macrophages to become antitumor effector macrophages, e.g. using an agonistic anti-CD40 antibody, or histidine-rich glycoprotein (HRG)), and/or aiming at activation of their antitumour activities (e.g. by stimulating antibody-dependent cellular toxicity (ADCC) or by stimulating antibody-dependent cellular phagocytosis (ADCP)). By inhibition of the IL-10 signaling (suppressing the tumor-promoting macrophage phenotype), and activation of macrophage and dendritic cells via TLR9 (sign of the antitumor macrophage phenotype), macrophages with the tumor-promoting macrophage phenotype could be skewed to cells with the antitumor macrophage phenotype (Vasievich et al. 2011, Mol Pharmaceutics 8:635-641; Table 1 herein lists a number of genes and strategies).

Neutralization of podoplanin-positive macrophages implies several possible levels of neutralization, e.g., at least 20%, at least 30%, at least 40%, at least 50%, at least 60%, at least 70%, at least 80%, at least 90%, at least 95% or even 100% neutralization. The nature of the neutralizing compound is not vital/essential to the invention as long as the podoplanin-positive macrophages are (partially) neutralized such as to treat or inhibit tumor metastasis and/or tumor-induced peritumoral lymphangiogenesis.

Further targeting moieties, other than podoplanin, can be identified experimentally, as will be described hereafter. By means of subtractive phage biopanning against whole cells, Cieslewicz et al. 2013 (Proc Natl Acad Sci USA 110:15919-15924) identified a peptide (M2pep) binding specifically to tumor-associated macrophages. The peptide was subsequently conjugated to a pro-apoptotic peptide (KLA, an all-D amino acid peptide; Ellerby et al. 1999, Nat Med 5:1032-1038). The fusion peptide selectively reduced the M2-like tumor-associated macrophages in mice, and delayed mortality of mouse with subcutaneously implanted CT26 cells (colon carcinoma). Using a phage screening procedure/screening of phage-displayed peptide libraries, Laakkonen et al. 2002 (Nat Med 8:751-755) identified the cyclic nonapeptide LyP-1 as targeting moiety to tumor cells, tumor lymphatics and tumor-associated macrophages. Yan et al. 2012 (J Control Release 157:118-125) discuss nanoparticles modified with LyP-1 and demonstrated targeted delivery of drug-loaded LyP-1-conjugated and PEGylated liposomes having the same homing specificity as LyP-1. LyP-1 is binding to the p32/gC1q receptor present on these tumor-associated cells. The same nonapeptide was earlier used to target microbubbles sensitive to ultrasound. Ultrasonic tumor molecular imaging or drug-delivery and therapy was therewith in reach and combines targeted delivery of the microbubbles with targeted delivery of the cargo contained in the microbubble (Li et al. 2009, Proceedings of 31st Annual International Conference of the IEEE Eng Med Biol Sci Minneapolis, Minn., USA, Sep. 2-6, 2009; 463-466).

Such strategies can likewise be applied to podoplanin-positive macrophages as described herein to identify peptides (targeting moieties) binding specifically to them on a surface component different from podoplanin itself. Other strategies include those based on cell sorting/flow cytometry/microfluidic chips as techniques to detect surface components unique to, enriched on, or specific for podoplanin-positive macrophages. The enrichment or specificity in this context is meant that the ratio of the amount of surface component (other than podoplanin) on podoplanin-positive macrophages over the amount of the same surface component on podoplanin-negative macrophages is higher than 1 (e.g. any number between 1 and 100-fold higher, or e.g. any number between 1 and 10-fold higher, e.g. 1.1, 1.2, 1.5, 1.7, 1.8, 2.0, 2.2, 2.5, 2.7, 3.0 fold higher), and sufficiently high to ensure sufficient selectivity of the podoplanin-positive macrophage neutralizing compound, preferably under in vivo conditions.

Further targeting possibilities, other than podoplanin, can be derived from the efforts that have already been invested in targeting a cargo specifically to tumor-associated macrophages. As indicated above, one way of visualizing sentinel lymph nodes relies on binding of a dye to endogenous albumin, which targets the dye to the lymph node. This “albumin-hitchhiking” process has been exploited to design amphiphilic “amph-vaccines” with increased ratio of lymph node accumulation over systemic dissemination. The amph-vaccines comprise an antigen or adjuvant cargo linked to a lipophilic albumin-binding tail by a solubility-promoting polar polymer chain. This was mimicked by administration of a conjugate of the antigen or adjuvant cargo directly to serum albumin (Liu et al. 2014, Nature 507:519-522). Including an albumin-binding moiety in the podoplanin-targeting moiety thus would be expected to increase selectivity of a podoplanin-positive macrophage neutralizing compound.

As indicated above, the macrophage mannose receptor (MMR or CD206) is enriched on podoplanin-positive macrophages. This receptor was targeted with a mannose-conjugated chlorin (M-chlorin) photosensitizer to bind mannose receptors expressed on tumor-associated macrophages. M-chlorin photodynamic therapy induced apoptosis of both cancer cells and tumor-associated macrophages (Hayashi et al. 2015, Mol Cancer Ther 14:452-460); systemic macrophages loaded with M-chlorin are spared due to being left inactive, hence not disturbing systemic immune function. In photodynamic therapy (PDT) light irradiation of a photosensitive molecule (photosensitizer, PS) in the presence of oxygen generates reactive oxygen species (ROS) which are cytotoxic. Cell ablation caused by PS after light exposure is spatially limited since ROS are short-lived/short-ranged species. Specificity to tumor-associated macrophages thus is obtained by molecular targeting combined with localized activation of a prodrug to produce localized cytotoxicity. A variant to this strategy was reported by Zhang et al. 2016 (Biomaterials 84:1-12) who designed a photoimmunotherapy compound in which a near-infrared phthalocyanine dye (IRDye700) is linked to a CD206-targeting antibody; in vivo efficacy in a sorafenib-resistant tumor model in mouse was demonstrated. These confirm that, in view of the enrichment of MMR on podoplanin-positive macrophages, MMR is a target that would add to the specificity of a podoplanin-positive macrophage neutralizing compound targeting podoplanin. A photosensitizer could be conjugated to such compound targeting podoplanin and targeting a second molecule (such as MMR or albumin) to allow specific ablation of podoplanin-positive macrophages. These reports also render it plausible that similar results would be obtained with other surface molecules unique to, enriched on, or specific for podoplanin-positive macrophages.

Ben-Nun et al. 2015 (Theranostics 5:847-862) developed a photosensitizer linked to a “quenched activity-based probe” (qABP) that allows for combined tumor detection and macrophage-targeted treatment. The specificity resides in activation by cathepsin (TAM-expressed cysteine cathepsins B, L and 5; releases the quencher moiety and renders the photosensitizer activatable), the activity of which is high in the tumor environment. The photosensitizer comprising probe part then covalently binds to cathepsin (unbound PS-qABP is not activated, therewith reducing background signal). This enables tumor detection, therewith aiding in directing the subsequent light-activation step of the photodynamic therapy. Such qABP is an alternative modification of a compound targeting podoplanin and optionally targeting a second molecule (such as MMR or albumin) present on podoplanin-positive macrophages in order to allow specific ablation of podoplanin-positive macrophages.

The challenges to target nanomedicines to tumor-associated macrophages have been reviewed by e.g. Andon et al. 2017, Semin Immunol, http://dx.doi.org/10.1016/j.smim.2017.09.004). Manipulation of size and composition (e.g. incorporating molecules involved in immune processes) of nanoparticles may enhance their uptake by/and modulate the response of immune-related cells. Manipulation of composition (passive targeting) and/or incorporation of targeting moieties (active targeting) increases specificity towards the target cells (reviewed by Dacoba et al. 2017, Semin Immunol http://dx.doi.org/10.1016/j.smim.2017.09.007). Nanoparticle size is decisive for the kinetics of nanoparticle migration through lymphatic vessels: nanoparticles with a diameter of less than 5-nm easily enter the bloodstream, whereas those with a diameter of over 100-nm remain at the injection site and do not move into lymphatic vessels. Larger particles with diameters ranging from 500 to 2000 nm are carried into lymph nodes by dendritic cells. Nanoparticles with a diameter of 15-70-nm seem optimal for rapid entry into lymphatic vessels and migration into lymph nodes (reviewed by Kim et al. 2017, Biomaterials 130:56-66).

A tri-block polymer nanoparticle capable of targeting TAMs for nucleotide delivery was designed by Ortega et al. 2015 (Nanoscale 7:500-510): a core comprised of a hydrophobic, pH responsive block that triggers endosomal escape and cytoplasmic delivery of the siRNA; a second block being a poly (DMAEMA) polymer with a polycationic charge; and a distal, azide-presenting block serves as a modular platform for further functionalization with targeting ligands or other biomolecules of interest. The second block attracts polyanionic oligonucleotides within the particle and serves to carry and protect siRNA for delivery to a target cell. Finally, the surface of these nanoparticles was coated with a mannose ligand in order to target tumor-associated macrophages via the mannose receptor (MMR or CD206). These mannose-coated nanoparticles were demonstrated to deliver siRNA in vivo specifically and effectively to tumor-associated macrophages. Zhu et al. 2013 (Mol Pharm 10:3525-3530) reported a mannose-modified nanoparticle platform for efficient targeting of a drug cargo to MMR on tumor-associated macrophages. The platform is built around inclusion of acid-sensitive PEG to the nanoparticles, which is shed only in the acidic tumor microenvironment (while uptake by macrophages in healthy organs or tissues is reduced due to PEG shielding at neutral pH). Both approaches are in line with the results of the targeted photodynamic therapies or photoimmunotherapies described above and again confirm the suitability of MMR as a tumor-associate macrophage targeting moiety. On the other hand, in the context of the current invention, another manner to increase specificity of such nanoparticles would be linkage to a compound targeting podoplanin an optionally targeting a second molecule (such as albumin) present on and enriched on the podoplanin-positive macrophages. The drug cargo (e.g. siRNA, small molecules, antibodies) of the nanoparticles could (in addition) serve as a podoplanin-positive macrophage neutralizing compound.

Huang et al. 2012 (J Control Release 158:286-292) relied on the high levels of galactose-type lectin on tumor-associated macrophages for targeted delivery of oligodeoxynucleotides (CpG, anti-IL-10 and anti-IL-10RA) complexed with galactosylated cationic dextran. An additional modification consisted of including the pH-sensitive PEG-histidine-modified alginate (PHA) in the complex. This modification adds to tumor-associated macrophage homing specificity as the dextran-oligodeoxynucleotide complex is released only in the acidic tumor environment. In the context of the current invention, another manner to increase specificity of oligodeoxynucleotides complexed with galactosylated cationic dextran would be linkage to a compound targeting podoplanin an optionally targeting a second molecule (such as MMR or albumin) present on and enriched on the podoplanin-positive macrophages. The oligodeoxynucleotides of the complex could (in addition) serve as a podoplanin-positive macrophage neutralizing compound.

A known phenomenon occurring when nanoparticles are contacted with biological fluids is the formation of a long-lived “protein corona” (reviewed by Barbero et al. 2017, Semin Immunol, http://dx.doi.org/10.1016/j.smim.2017.10.001) which can cover the targeting/homing ligands on the nanoparticle surface, therewith reducing the nanoparticle targeting/homing capabilities. In solving this problem occurring with upconversion nanoparticles (UCNPs; which convert light from the near-infrared (NIR) range to the visible range, and can be applied in vivo fluorescence imaging), Rao et al. 2017 (ACS Appl Mater Interfaces 9:2159-2168) reconstructed into vesicles cell membranes from red blood cells, which were then coated onto UCNPs. After confirming the avoidance of corona formation, the UCNPs coated with red blood cell membrane vesicles were functionalized for targeting to tumor cells by coupling of folic acid (targeting the folate receptor expressed on tumor cells). Other nanoparticles (such as with a therapeutic payload or with a payload aimed at neutralizing podoplanin-positive macrophages) could be protected from protein corona formation in a similar way, and, in the context of the current invention, could be targeted to podoplanin-positive macrophages by linkage to a compound targeting podoplanin and optionally targeting a second molecule (such as MMR or albumin) present on and enriched on the podoplanin-positive macrophages.

The above illustrates the availability of a number of candidate targeting moieties different from podoplanin to increase the specificity of podoplanin-positive macrophage neutralizing compounds and in the same time provided some examples of compounds that could serve such purpose. The examples and results available for CD206 render it plausible that similar results can be obtained with e.g. CD86 or CD204.

On the other hand, bispecific or multispecific molecules in themselves may be sufficient to neutralize podoplanin-positive macrophages (e.g. in neutralizing the capability of binding of podoplanin-positive macrophages to lymph vessel or lymph node cells), wherein such bispecific or multispecific molecules are binding to podoplanin on tumor-associated macrophages at one hand, and are at the other hand binding to a targeting moieties different from podoplanin and unique to, enriched on, or specific for podoplanin-positive macrophages. Examples of such bispecific molecule include bispecific antibodies binding to podoplanin and e.g. MMR/CD206, CD86, or CD204. Multispecific molecules include those binding to podoplanin and to two of e.g. MMR/CD206, CD86, or CD204; or those binding to podoplanin, to one of e.g. MMR/CD206, CD86, or CD204, and to human albumin. Other bi- or multi-specific molecules include those at the one hand binding to podoplanin and at the other hand including one or more of mannose, a galactosylated carrier (such as galactosylated dextrin), M2pep or LyP-1.

Several of the podoplanin-positive macrophage targeting moieties can be combined in one molecule as long as binding of the combined moieties to the macrophage-targets is possible, e.g. by including flexible linkers between the targeting moieties. A specific example is the tandem combination of several immunoglobulin single variable domains (ISVD) each binding to a different target. ISVDs or single-domain VHH antibody fragments (also named nanobodies) are derived from the heavy-chain-only antibodies found in camelid species (Hamers-Casterman et al. 2003, Nature 363: 446-448). Besides a podoplanin-targeting ISVD, such tandem combination could for instance comprise an ISVD against CD206/MMR and/or an ISVD against albumin. Exemplary immunoglobulin single variable domains directed against human macrophage mannose receptor (MMR) have been disclosed in WO 2013/174537 and in WO 2017/158436. Exemplary immunoglobulin single variable domains directed against human albumin have been disclosed in EP17182200.00. Such tandem combinations have been successfully produced before by interspersing the different ISVDs with flexible (GlyₙSer)ₘ linkers (n and m being integers)—just one example is a trispecific molecule combining ISVDs targeting MMP8, TNFR1, and albumin (PCT/EP2017/076427). The macrophage-targeting immunoglobulins do not need to be ISVDs. Non-limiting examples of other suitable immunoglobulins are (monoclonal) antibodies or antigen-binding fragments thereof, alpha-bodies, nanobodies, intrabodies (antibodies binding and/or acting to intracellular target; this typically requires the expression of the antibody within the target cell, which can be accomplished by gene therapy). None-limiting examples of other suitable molecules include aptamers, DARPins, affibodies, affitins, anticalins, monobodies, bicyclic peptide (as described in e.g. WO 2004/077062 wherein e.g. 2 peptide loops are attached to an organic scaffold; phage-display screening of such peptides has proven to be possible in e.g. WO 2009/098450).

Any of the above compounds targeting podoplanin-positive macrophages may further comprise a therapeutic payload as described above. In specific cases, such payload may be in the form of a prodrug. For instance, Dubowchik et al. 2002 (Bioconjugate Chem 13:855-869) showed that the N-capped (maleimidocaproyl as capping group) dipeptide (Phe-Lys) linked to doxorubicin could be included in an immunoconjugate with an antibody (via maleimidocaproyl) to result in a cathepsin 6-labile prodrug preferentially activated in the vicinity of cancer cells. Walker et al. 2002 (Walker et al. 2004, Bioorg Med Chem Lett 14:4323-4327) successfully followed the same strategy as Dubowchik et al. 2002 but with another cytotoxic drug, i.e. tallysomycin S10b.

### Detectable Label or Detectable Moiety

A detectable label or moiety in general refers to a moiety that emits a detectable signal or is capable of emitting a detectable signal upon adequate stimulation, and is detectable by any means. For molecules comprising a detectable label to be administered to a mammal (such as a human), said detection is preferably by a non-invasive means, once inside the mammalian body. Furthermore, the detectable moiety may allow for computerized composition of an image, as such the detectable moiety may be called an imaging agent. Detectable moieties include reaction products of enzymes (e.g. alkaline phosphatase, horseradish peroxidase), phosphorescence emitters (such as produced by enzymatic action, but not limited thereto), fluorescence emitters (such as produced by enzymatic action, but not limited thereto), positron emitters, radioemitters, etc.

Measuring the amount of detectable moiety/imaging agent is typically done with a colorimetric device; a device capable of measuring one or more of absorbance, fluorescence intensity, luminescence, time-resolved fluorescence, or fluorescence polarization; a device counting radioactivity or determining radiation (which can be of photonic nature, e.g. fluorescent or phosphorescent) density or radiation concentration; or by capture on a suitable substrate (e.g. membranes or beads to which proteins or nucleic acids are bound and capable of binding a colorimetric agent, fluorescent emitters, . . . ; e.g. X-ray films sensitive to radiation), etc. When a signal is determined in multiple zones of a 2- or 3-dimensional specimen, the counted, measured or determined signal can be transformed into a 2- or 3-dimensional image. Depending on the nature of the emission by the detectable moiety, it may be detectable by techniques such as PET (positron emission tomography), SPECT (single-photon emission computed tomography), fluorescence imaging, fluorescence tomography, near infrared imaging, near infrared tomography, optical tomography, etc.

Examples of radioemitters/radiolabels include ⁶⁸Ga, ^{110m}In, ¹⁸F, ⁴⁵Ti, ⁴⁴Sc, ⁴⁷Sc, ⁶¹Cu, ⁶⁰Cu, ⁶²Cu, ⁶⁶Ga, ⁶⁴Cu, ⁵⁵Ca, ⁷²As, ⁸⁶Y, ⁹⁰Y, ⁸⁹Zr, ¹²⁵I, ⁷⁴Br, ⁷⁵Br, ⁷⁶Br, ⁷⁷Br, ⁷⁸Br, ¹¹¹In, ^{114m}In, ¹¹⁴In, ^{99m}Tc, ¹¹C, ³²Cl, ³³Cl, ³⁴Cl, ¹²³I, ¹²⁴I, ¹³¹I, ¹⁸⁶Re, ¹⁸⁸Re, ¹⁷⁷Lu, ⁹⁹Tc, ²¹²Bi, ²¹³Bi, ²¹²Pb, ²²⁵Ac, ¹⁵³Sm, and ⁶⁷Ga. Fluorescence emitters include cyanine dyes (e.g. Cy5, Cy5.5, Cy7, Cy7.5), indolenine-based dyes, benzoindolenine-based dyes, phenoxazines, BODIPY dyes, rhodamines, Si-rhodamines, Alexa dyes, and derivatives of any thereof.

Many of the radionuclides have a metallic nature and are typically incapable of forming stable covalent bonds with proteins or peptides. One solution is to label proteins or peptides with radioactive metals by means of chelators, i.e. multidentate ligands, which form non-covalent compounds, called chelates, with the metal ions. The detectable moiety, may itself be comprised in a prosthetic group and the prosthetic group may be linked to the polypeptide through a chelator or conjugating moiety such as a cyclooctyne comprising a reactive group that forms a covalent bond with an amine, carboxyl, carbonyl or thiol functional group on a polypeptide.

### Other Definitions

The present invention is described with respect to particular embodiments and with reference to certain drawings but the invention is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated. For example “a nucleotide” or “an oligonucleotide” is not to be understood as a single nucleotide molecule or a single oligonucleotide molecule, respectively, but rather as a collection of the same nucleotide molecules or a collection of the same oligonucleotide molecules, respectively.

Furthermore, the terms first or i), second or ii), third or iii), and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

Unless specifically defined herein, all terms used herein have the same meaning as they would to one skilled in the art of the present invention. Practitioners are particularly directed to Sambrook et al., Molecular Cloning: A Laboratory Manual, 2^{nd} ed., Cold Spring Harbor Press, Plainsview, N.Y. (1989); and Ausubel et al., Current Protocols in Molecular Biology (Supplement 47), John Wiley & Sons, New York (1999), for definitions and terms of the art. The definitions provided herein should not be construed to have a scope less than understood by a person of ordinary skill in the art.

“Treatment”/“treating” refers to any rate of reduction or retardation of the progress of the disease or disorder compared to the progress or expected progress of the disease or disorder when left untreated. More desirable, the treatment results in no/zero progress of the disease or disorder (i.e. “inhibition” or “inhibition of progression”) or even in any rate of regression of the already developed disease or disorder. “Suppressing” can in this context be used as alternative for “treating”.

The group of mammals includes, besides humans, mammals such as primates, cattle, horses, sheep, goats, pigs, rabbits, mice, rats, guinea pigs, llama's, dromedaries and camels.

The methods described above in general may comprise the administration of a podoplanin-positive macrophage neutralizing compound to a mammal in need thereof, i.e., harboring a tumor, cancer or neoplasm in need of treatment, in particular in need of treating or inhibiting metastasis and/or tumor-induced lymphangiogenesis. In general a (therapeutically) effective amount of a podoplanin-positive macrophage neutralizing compound is administered to the mammal in need thereof in order to obtain the described clinical response(s). The (therapeutically) effective amount of podoplanin-positive macrophage neutralizing compound will depend on many factors such as route of administration and tumor mass and will need to be determined on a case-by-case basis by the physician.

It is to be understood that although particular embodiments, specific configurations as well as materials and/or molecules, have been discussed herein for cells and methods according to the present invention, various changes or modifications in form and detail may be made without departing from the scope and spirit of this invention. The following examples are provided to better illustrate particular embodiments, and they should not be considered limiting the application. The application is limited only by the claims.

The content of the documents cited herein are incorporated by reference.

### EXAMPLES

### Example 1. Expression of Podoplanin in Tumor-Infiltrating Leukocytes

Tumor-bearing mice were sacrificed by cervical dislocation and tumors were harvested. 4T1 breast tumours were minced in RPMI medium containing 0.1% collagenase type I and 0.2% dispase type I (Gibco) for 30 min at 37° C. and passed through a 70 and 40 μm cell strainer. After red blood cell lysis lysis, cells were resuspended in FACS buffer (PBS+FBS+EDTA). Single-cell suspensions were incubated for 15 min at 4° C. with Mouse BD Fc Block 1:100 in FACS buffer. The antibodies were added in the appropriate combinations.
A. TAMs: viable cells CD45+, CD11b+, F4/80+
B. Neuthrophils: viable cells, CD45+, CD11B+, Ly6G+
C. Monocyte-derived dendritic cells: viable cells, CD45+, CD11B+, CD11c+
D. Classical dendritic cells: viable cells, CD45+, CD11B−, CD11c+
E. NK cells: viable cells, CD45+, CD11B−, NKp46+
F. B cells: viable cells, CD45+, CD11B−, CD9+
G. Tc cells: viable cells, CD45+, CD11B−, CD8+
H. Th cells: viable cells, CD45+, CD11B−, CD4+

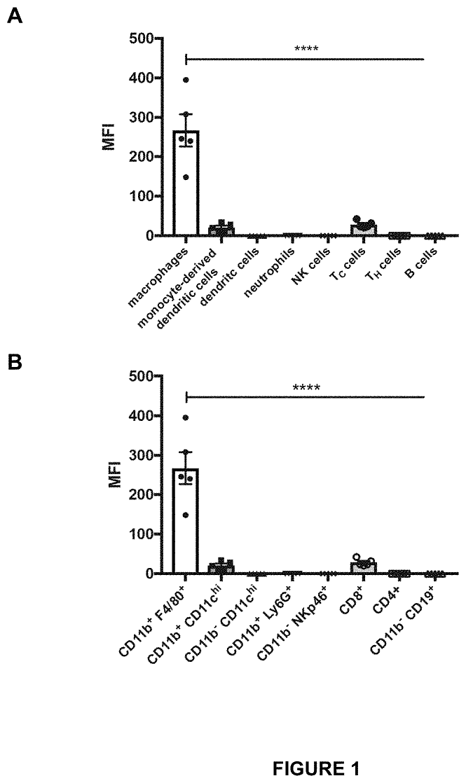

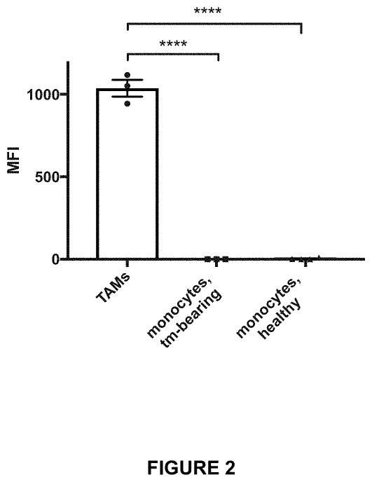

Tumor-associated macrophages (TAMs) can be virtually involved in all the phases of tumor progression. In this scenario, distinguished macrophage subsets/differentiation states are associated to specific biological processes and have been assigned with different functions. Among all the cells of the immune system, it was found that in orthotopic 4T1 breast tumors Pdpn was almost exclusively expressed in TAMs but not in other tumor-infiltrating leukocytes (TILs). F4/80+ skin macrophages or circulating (resident and inflammatory) monocytes from these tumor-bearing mice were also negative for Pdpn. This is illustrated in FIGS. 1 and 2.

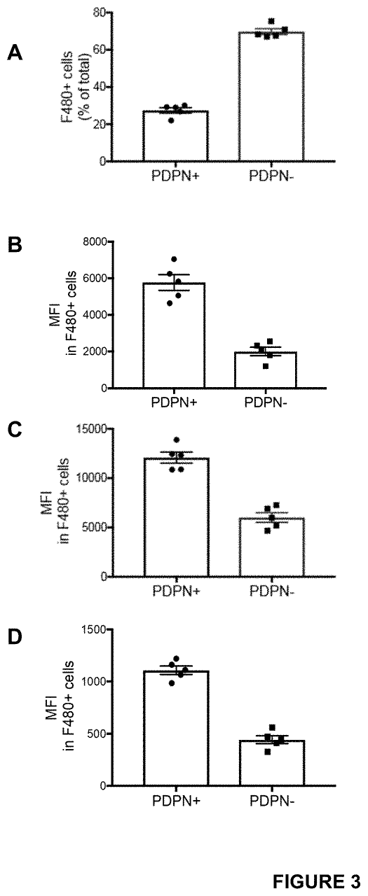

Among wild-type TAMs, around 30% is podoplanin-positive as determined by FACS-analysis (FIG. 3A). It was further analyzed whether podoplanin-expression on TAMs was associated with enriched expression of other macrophage markers. Initial FACS analysis indicated that for instance CD206 (macrophage mannose receptor), CD86 and CD204 seem to be expressed at significantly higher levels in podoplanin-positive macrophages (PDPN+ TAMs) compared to podoplanin-negative macrophages (PDPN− TAMs). These initial results are illustrated in FIG. 3B-D.

### Example 2. Analysis of Tumor Growth and Tumor Metastasis in Wild-Type Mice and in Mice with a Podoplanin-Deficient Hematopoietic System

Five- to 6-week-old C56BL/6 recipient female mice were irradiated with 7.5 Gy. Subsequently, 10⁷ bone marrow cells from the appropriate genotype (Pdpn+/+ or Pdpn−/−) were injected intravenously via the tail vein. Tumor experiments were initiated 5-6 weeks after bone marrow reconstitution.

Donor BM isolation: donor mice are sacrificed by cervical dislocation; femurs and tibias are removed (femurs are isolated completely, tibias are cut just above the heel); bones are collected in cold sterile RPMI complete+10% FBS on ice; in the laminar flow: bone marrow is flushed with RPMI complete+10% FBS with a 10 ml syringe with a 27-G needle; cells are collected by centrifugation (1250 rpm) for 10 min at 4° C.; cell pellet is resuspended in PBS; suspension is filtered through sterile cell strainer 40 μm; cells are counted and resuspended to inject.

All cancer cells were injected in the 2^{nd} bottom left nipple; 4T1: 1×10⁶ cells & mice sacrificed at day 24; EMT6.5: 1×10⁶ cells & mice sacrificed at day 30; E0771: 1×10⁶ cells & mice sacrificed at day 30.

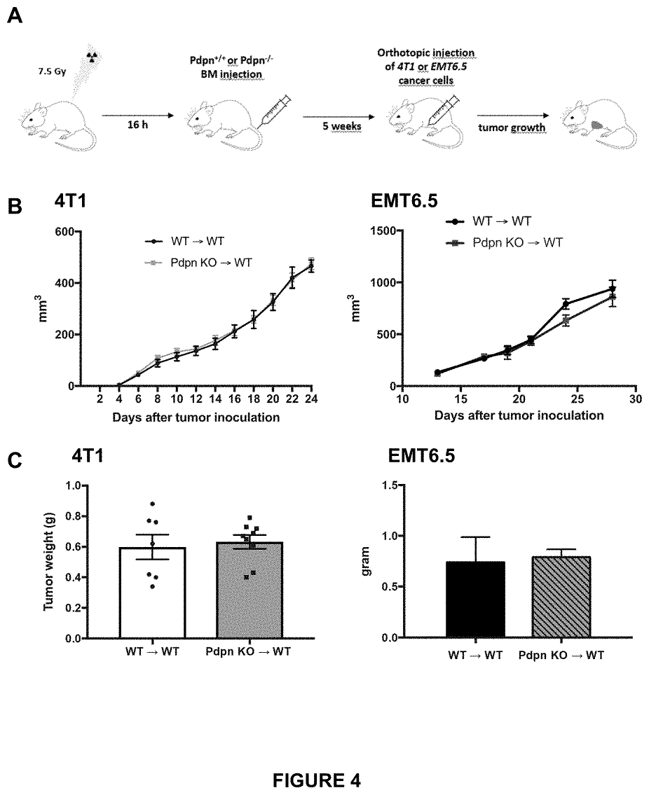

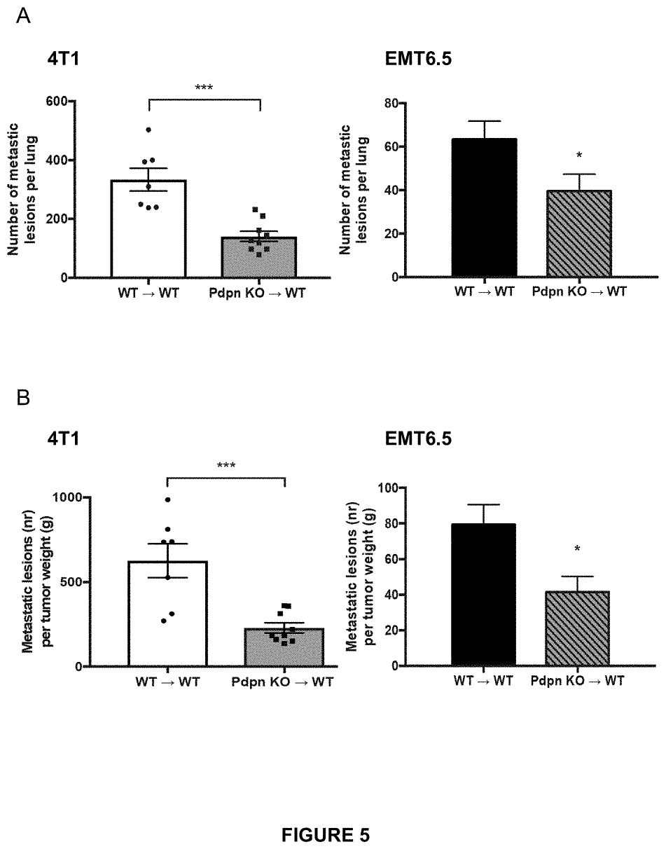

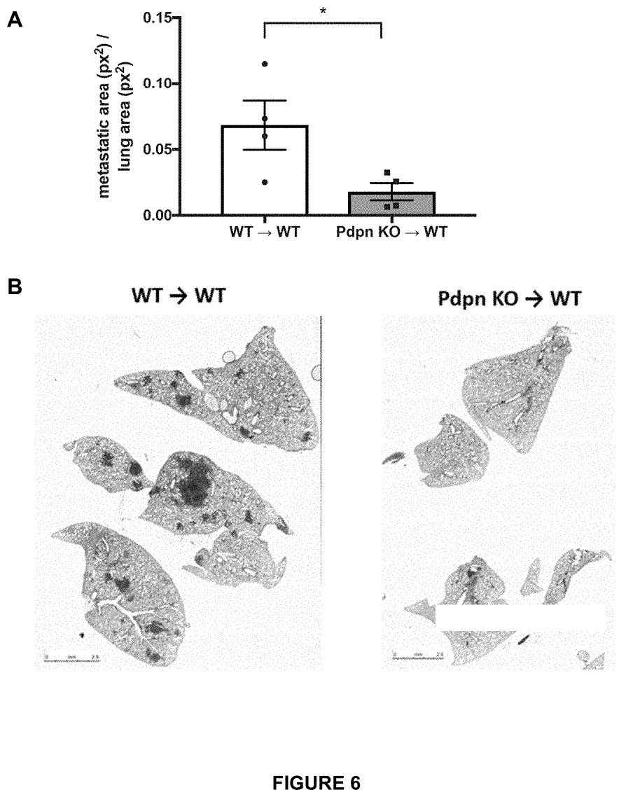

To study the role of podoplanin in TAMs, wild-type (WT) and podoplanin-knockout (Pdpn KO) bone marrow (BM) cells were used to reconstitute the immune system of lethally irradiated WT BalbC recipient mice thus generating WT→WT and Pdpn KO→WT chimeric mice. Five to six weeks after reconstitution, syngeneic 4T1 or EMT6.5 breast cancer cells were injected orthotopically in both WT→WT and Pdpn KO→WT chimeras (schematically shown in FIG. 4A). Despite comparable tumor growth (FIGS. 4 B-C), 24 days after cancer cell injection the number of pulmonary metastasis was lower in Pdpn KO→WT versus WT→WT mice in both breast cancer models, albeit somewhat more pronounced in the 4T1 model than in the EMT6.5 model (which is most likely explainable by the higher degree of variation observed in the EMT6.5 model) (FIGS. 5A-B). When calculating the metastatic area/lung area, a reduction of up to 60% of lung metastasis is observed in the 4T1 model (FIG. 6A)—the reduction of lung metastasis is illustrated by immunohistochemistry in FIG. 6B.

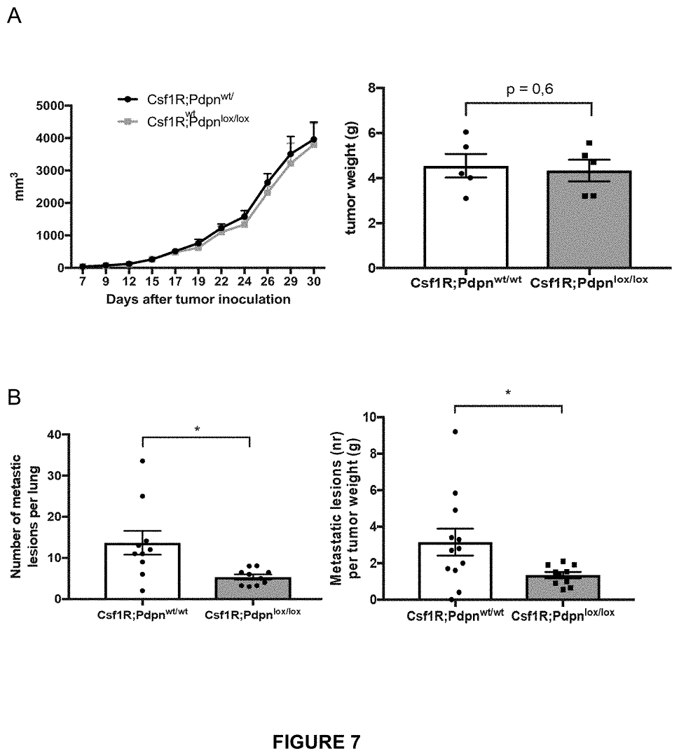

Similar results were obtained with another breast cancer model, E0771, in mice in which expression of podoplanin in Csf1R+/+ TAMs could be conditionally deleted upon induction with tamoxifen. More in particular, exon1 of the Pdpn gene was inserted with lox fragments on both ends. The Pdpn loxed mice were intercrossed for at least two generations with CsfR1:Cre in order to obtain CsfR1^{fl/fl} Cre-negative (WT) or CsfR1^{fl/fl} Cre-positive (KO) littermates for the specific promoter. Podoplanin deletion did not affect growth of the primary tumor (FIG. 7A) but lung metastasis was significantly reduced (FIG. 7B).

### Example 3. Analysis of Lymph Node Metastasis in Wild-Type Mice and in Mice with a Podoplanin-Deficient Hematopoietic System

Since 4T1 cells, similar to human breast cancer, spread through the lymphatics, the lymph node status and the histological features of tumor lymph vessels were analysed.

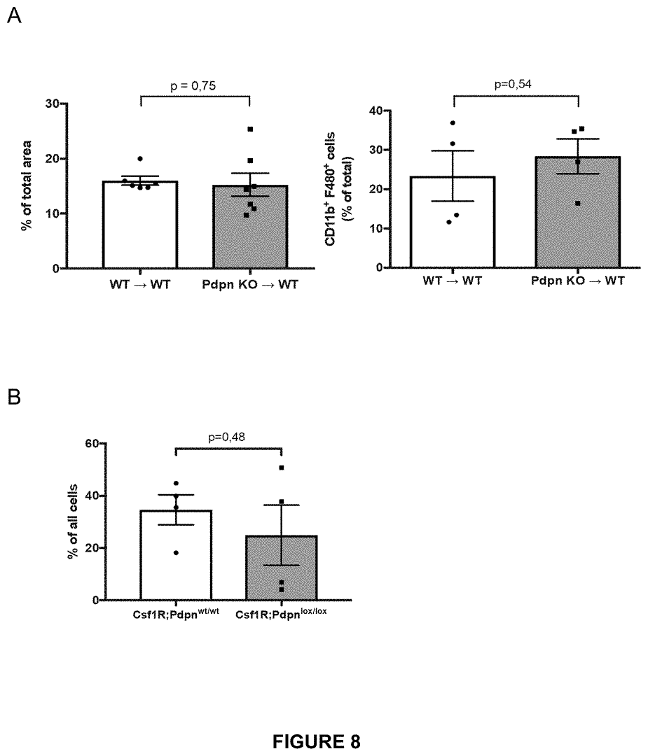

In a first step, it was assessed whether deletion of podoplanin in the TAMs altered their recruitment to tumors. Because of the expression of Pdpn mainly in TAMs, we sorted CD11b+F4/80+ macrophages from 4T1 tumors and measured the extent of Pdpn depletion in WT→WT and Pdpn KO→WT mice. TAMs from Pdpn KO→WT mice were depleted for Pdpn as shown by both qRT-PCR and FACS. This reduction did not impact on overall TAM density as assessed by either immunohistochemistry on tumor sections or FACS analysis on whole tumors. As shown in FIG. 8A, podoplanin deficiency did not alter recruitment of TAMs to the tumors. Similar results were obtained in the E0771 model by FACS analysis: podoplanin deficiency did not significantly alter recruitment of TAMs to tumor stroma (FIG. 8B).

However, it was clearly observed that the association of TAMs to the lymphatics within a distance of 25 μm was strongly impaired in absence of Pdpn. The aberrant localization in the peri-vascular space of Pdpn KO TAMs was not observed when focusing on the blood vessels. Although Pdpn was required for the association of TAMs to the lymphatic vessels, its deletion did not affect TAM phenotype as characterized by FACS and qRT-PCR for the expression of the main polarization markers and lymphangiogenic genes.

In a different breast cancer model, namely EMT6.5, Pdpn KO TAMs also had impaired peri-lymphatic distribution, diminished lymph vessel area and number, and reduced metastatic burden (despite unaltered tumor growth). Finally, Pdpn^{iΔMac} mice were generated in a C57BL/l6 background, lacking Pdpn specifically in TAMs after tamoxifen injection. Induction of Pdpn deletion 2 days before orthotopic implantation of syngeneic E0771 breast cancer cells also resulted in defective perilymphatic TAM localization, reduced lymphangiogenesis, and strong metastatic burden reduction with comparable tumor growth.

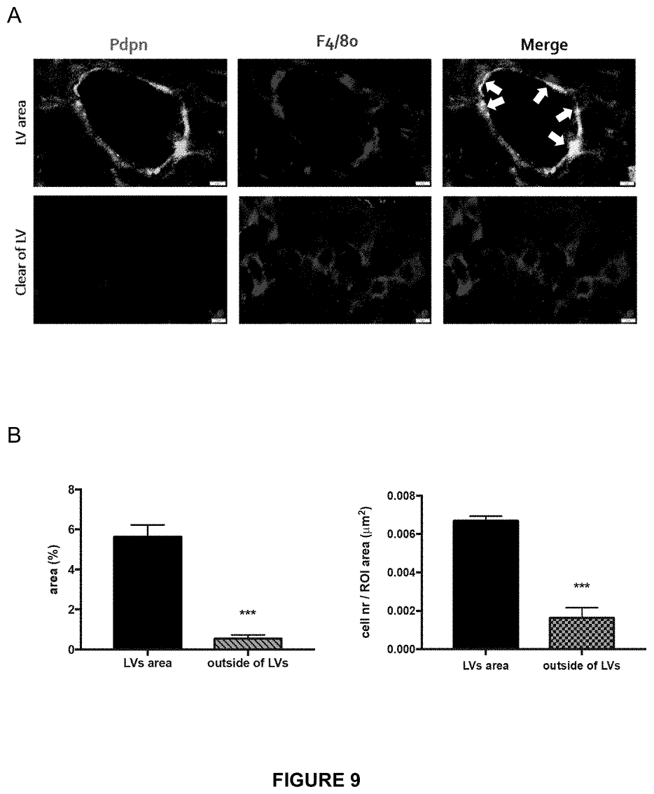

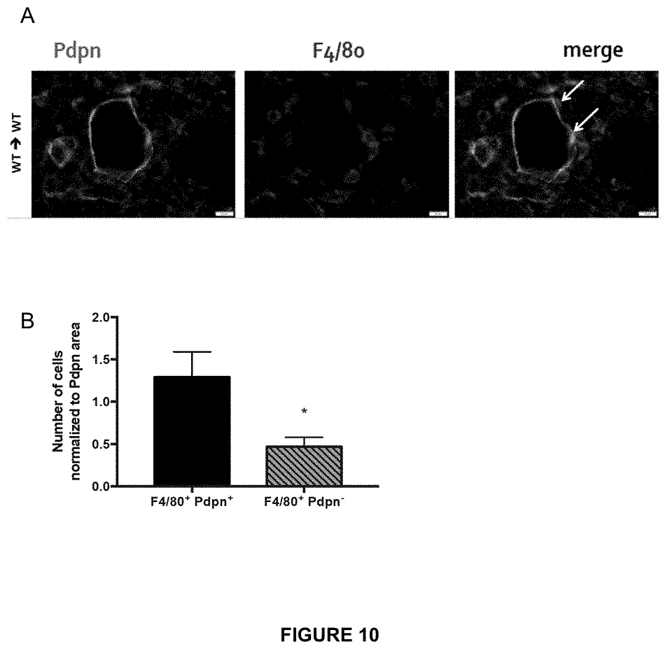

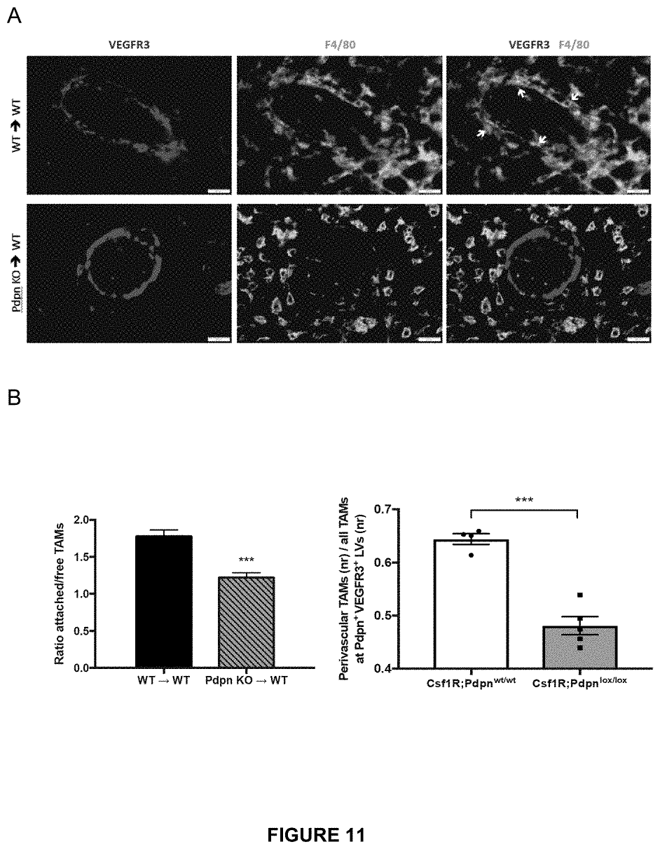

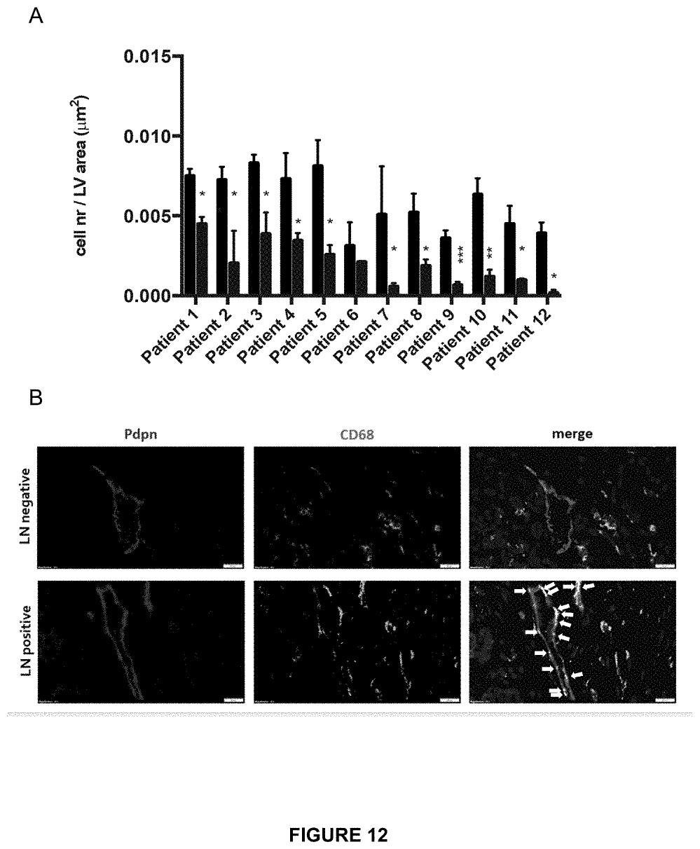

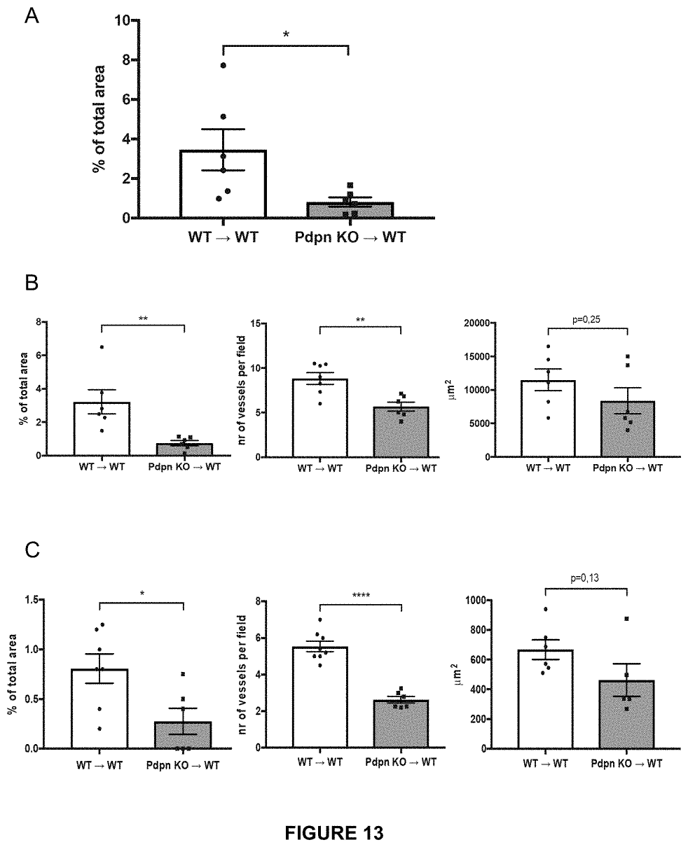

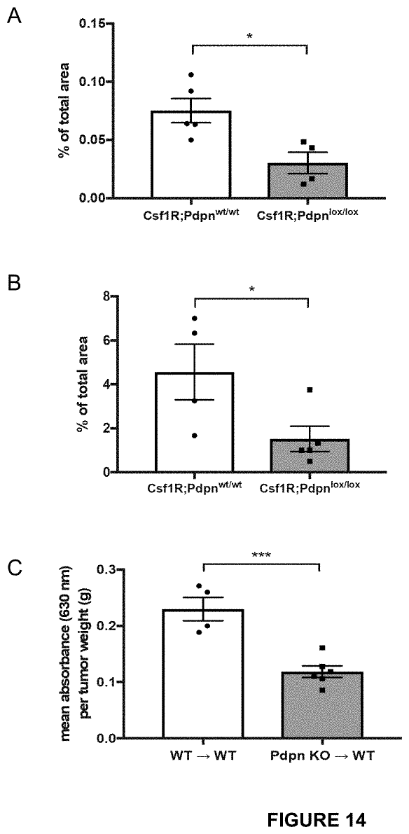

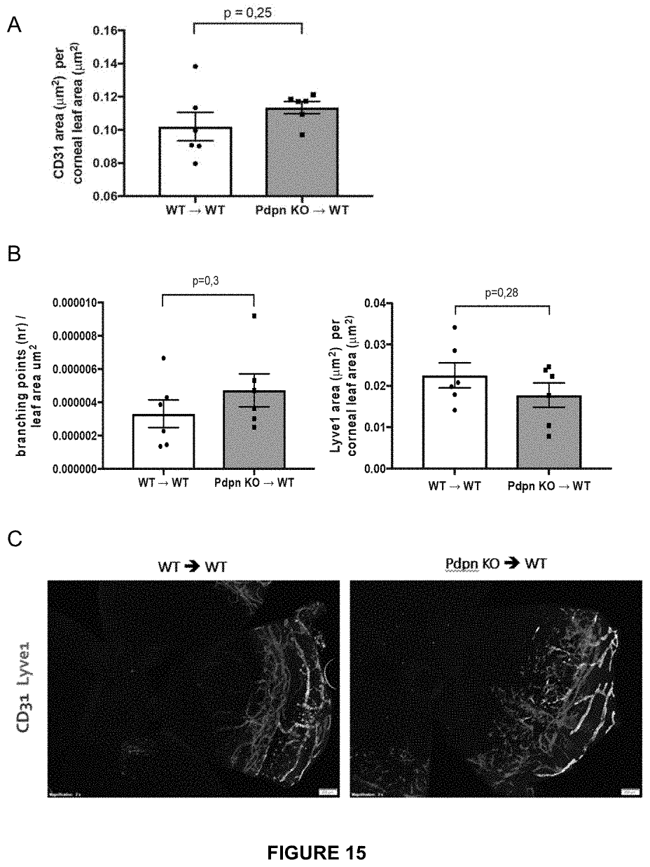

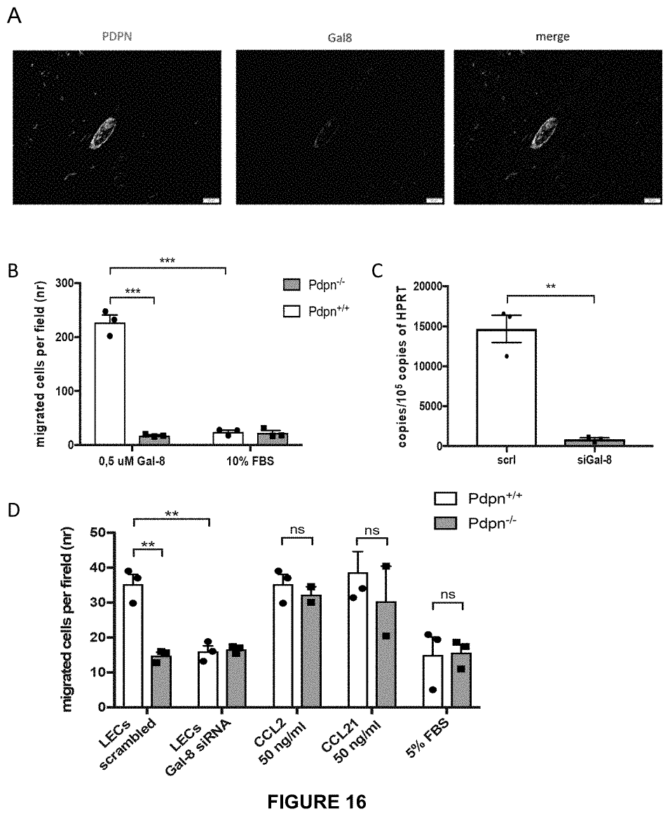

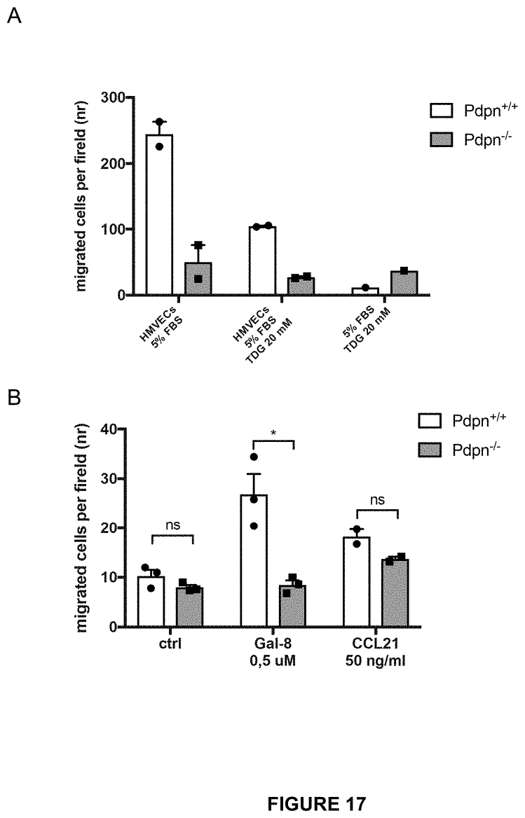

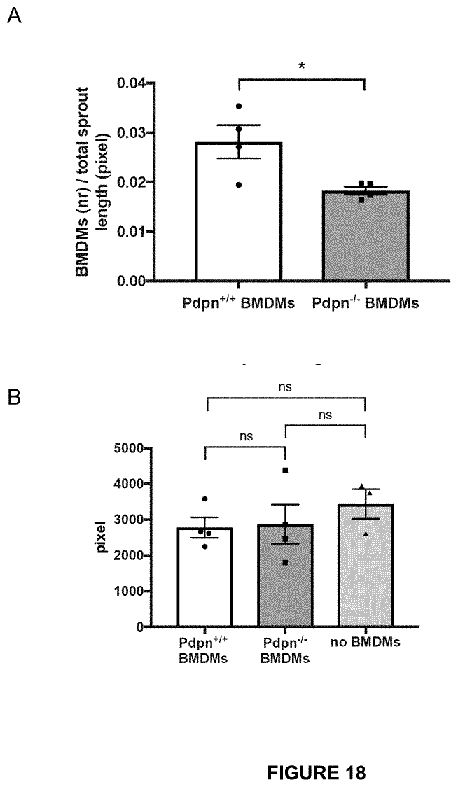

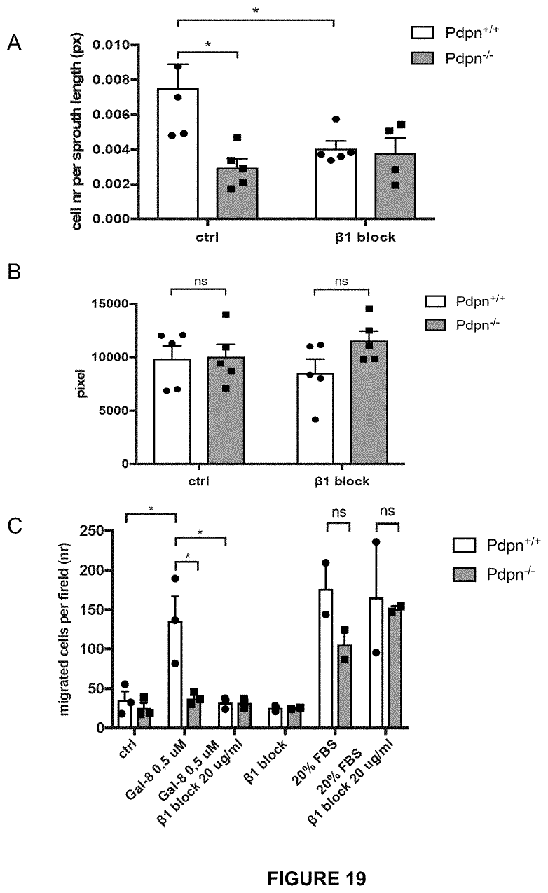

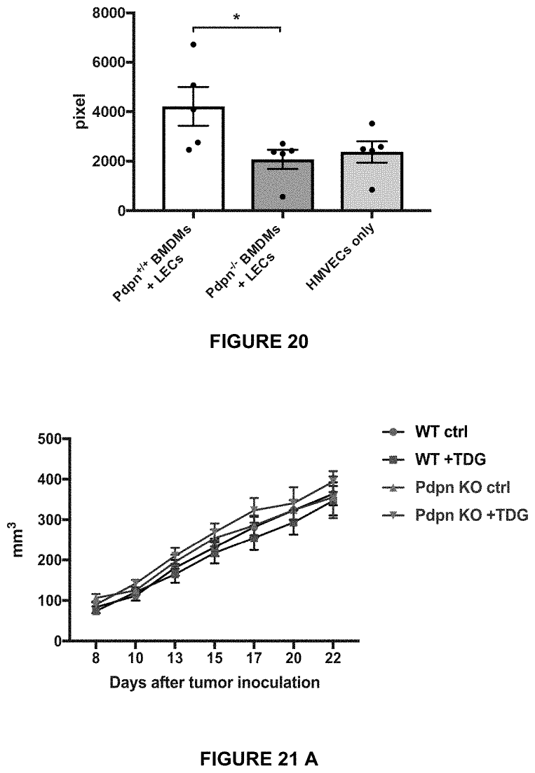

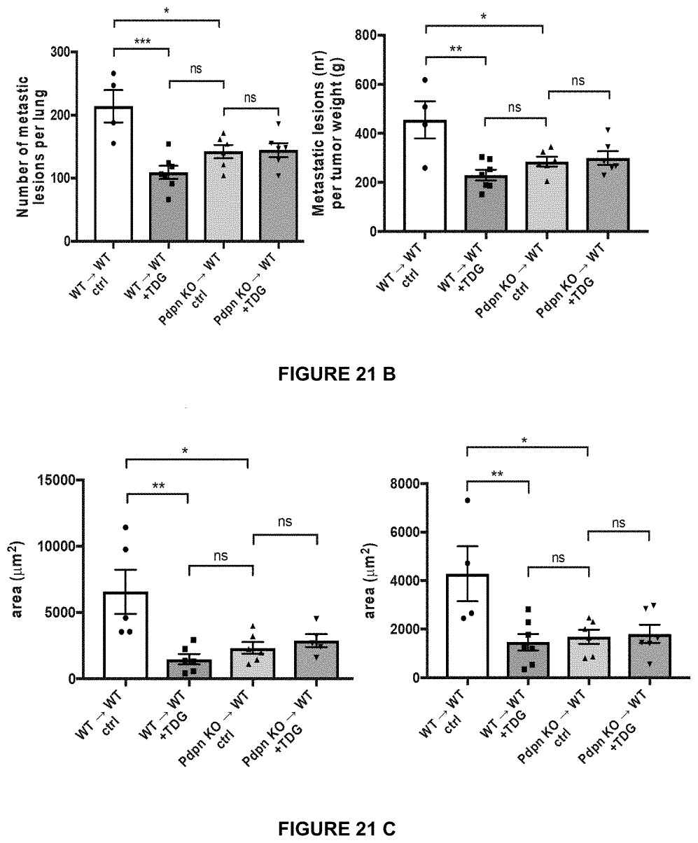

Thus, in focusing on the lymphatics, PDPN+ TAMs were located remarkably closer to the lymphatics in the 4T1 tumor model (FIG. 9A-B). Looking at perivascular TAMs, these were shown to be mainly podoplanin-positive (FIG. 10A-B). Finally, it was shown that PDPN+ TAMs interact more strongly with lymphatic endothelial cells compared to PDPN− TAMs, both in 4T1 and E0771 tumor models (FIG. 11A-B).

The mTmG transgenic line crossed with the inducible macrophage specific CSF1R:Cre-ERT deleter display TAMs in green-(EGFP+) while all other stromal components are red fluorescent (Tomato+). This model enabled us to further support the idea that, in 4T1 breast tumors, PDPN+ EGFP+ macrophages are more at the lymphatic side where they localize in the proximity or adhere to the lymphatic wall but they never integrate to (PDPN+ Tomato+) lymphatic vessels. Confocal imaging on thick 4T1 breast tumor sections further confirmed the localization of PDPN+ F4/80+ macrophages around (but not within) the lymphatic vessel wall.

### Example 4. Metastatic Positive Lymph Nodes of Human Breast Cancer are Positive for Podoplanin-Positive TAMs

In attempt to translate the findings in mice as described in Example 3 to human breast cancer patients, it was confirmed that PDPN+ but not PDPN− TAMs were present around lymphatic vessels. In a cohort of patients with bilateral tumors in which one was giving rise to ipsilateral lymph node metastasis while the contralateral tumor was lymph node negative for metastasis, it was established that the adhesion of PDPN+ TAMs to the lymphatics wall correlated with the presence of metastatic lymph nodes. This is illustrated in FIG. 12A-B.

### Example 5. Analysis of Peritumoral Lymphangiogenesis in Wild-Type Mice and in Mice with a Podoplanin-Deficient Hematopoietic System

It was found that the number of lymphatic vessels within the tumor was 50% lower in PDPN KO→WT versus WT→WT mice (FIG. 13 A-C). This was likewise observed in the E0771 tumor model (FIG. 14 A-B).

The impaired lymphangiogenesis was associated with decreased Evans Blue drainage from the tumors to the inguinal lymph nodes (FIG. 14C; 4T1 tumor model). Tumor blood vessel density and area as well as coverage and perfusion did not change.

In a different breast cancer model, namely EMT6.5, Pdpn KO TAMs also had impaired peri-lymphatic distribution, diminished lymph vessel area and number, and reduced metastatic burden (despite unaltered tumor growth). Finally, we generated Pdpn^{iΔMac} mice in a C57BL/l6 background, lacking Pdpn specifically in TAMs after tamoxifen injection. Induction of Pdpn deletion 2 days before orthotopic implantation of syngeneic E0771 breast cancer cells also resulted in defective perilymphatic TAM localization, reduced lymphangiogenesis, and strong metastatic burden reduction with comparable tumor growth.

Importantly, in a cornea wound/cauterization assay, WT→WT and Pdpn KO→WT chimeras displayed similar pathological angiogenesis and lymphangiogenesis (FIG. 15 A-C).

Overall, the findings as outlined in Examples 1-5 indicate that podoplanin on TAMs is required for the localization of TAMs around the lymphatics but it is dispensable for their differentiation state. By impairing TAM recruitment in the perilymphatic space, lymph vessel maintenance in Pdpn KO→WT mice is affected with consequent reduction of lymphatic metastasis.

### Example 6. Role of Galectin-8

In the BMDM/TAM migration assays, 1×10⁵ macrophages (labelled with calcein) were seeded in the top chamber. The bottom chamber contained a soluble attractant (0.5 μM Gal-8) or lymphatic endothelial cells (2×10⁵ cells) seeded 12 h prior to macrophage migration start. Gal-8 inhibitor TDG (20 mM) was added or not to the bottom chamber with LECs. Macrophages migrated for 4 h. Afterwards, the medium was aspirated from the transwells, the transwells were fixed with 4% PFA for 20′ and mounted onto glass slides with Fluoromount.

BMDMs used in migration assays are obtained from the bones of donor WT or PDPN KO mice (see Example 2), cultured for 7 days with L929 medium (containing mCSF, macrophage colony stimulating factor). TAMs were isolated by FACS sorting (as CD11b+ F480+ cells).

Galectin-8 (Gal-8), a secreted glycan-binding protein specifically expressed by lymph vessels that modulates processes of cell adhesion and migration is known to interact with podoplanin. First, the expression of Gal8 on 4T1 tumor sections was checked and it was found that this protein is specifically expressed by tumor lymphatics (FIG. 16A). Second, the binding of Gal8 to Pdpn WT bone-marrow derived macrophages (BMDMs) was validated, also expressing Pdpn at the RNA and protein level. Gal8 binding was severely impaired in Pdpn KO BMDMs. Finally, when measuring the migratory capacity of WT and Pdpn KO macrophages, it was noticed that WT BMDMs passed more efficiently through a 8 μm-pore membrane when recombinant (soluble) Gal8 was added to the medium. In contrast, Pdpn KO BMDMs lost completely this response to Gal8 (FIG. 16B).

A further migration assay was set up wherein the upper chamber contained BMDMs and the lower chamber cultured lymphatic endothelial cells (LECs). Only PDPN+ BMDMs migrated towards factors released by the LECs. When the LECs were silenced for Gal-8 expression (by means of siRNA, FIG. 16C), migration was abolished. CCL2 and CCL21 known to induce macrophage migration, and 5% FBS, were included as positive controls and attract PDPN+ and PDPN− BMDMs to equal extents (FIG. 16D). As an alternative for Gal-8 silencing, the pan-galectin inhibitor thiodigalactoside (TDG) was demonstrated to suppress migration of PDPN+ BMDMs to LECs (FIG. 17A). Finally, TAMs were sorted from 4T1 tumors and shown to be attracted by soluble Gal-8 in a podoplanin-dependent manner, this in contrast to migration towards CCL21 (FIG. 17B).

In the same assay, addition of another well-known Pdpn ligand, CLEC2, did not affect the migration of Pdpn WT or Pdpn KO BMDMs. As a further positive control, it was proven that the migratory capacity towards high FBS concentration (20%) was comparable for both WT and KO cells.

The effect of the galectin-8 inhibitor TDG on 4T1 tumor growth and metastasis was assessed. TDG was injected intramurally 3 times a week (Monday-Wednesday-Friday) starting from day 6 on, in a concentration of 120 mg per kg of body weight. Administration of TDG did not influence primary tumor growth, independent of reconstitution with PDPN+ or PDPN− TAMs (FIG. 21A). On the other hand, TDG-treatment reduced lung metastasis of mice reconstituted with PDPN+ TAMs, but did not further reduce metastasis already obtained in mice reconstituted with PDPN− TAMs (FIG. 21B). The observed reduced lung metastasis with TDG correlated with reduced peritumoral lymphangiogenesis (FIG. 21C).

### Example 6. Role of Integrin B1 (CD29)

Prior to migration assay, BMDMs were incubated 20 min with a integrin beta 1-blocking antibody (clone HMβ1-1 from Biolegend) on ice in PBS (1 min cells in 100 ul PBS with 20 ug/ml antibody) or just in PBS (as a control). Then BMDMs were washed once and resuspended in migration assay medium.

In an attempt to assess the relevance of macrophage-born Pdpn in the interaction with the lymphatic endothelium, adherent WT and Pdpn BMDMs on top of in vitro formed lymphatic capillaries were counted. Deletion of Pdpn in macrophages clearly affected their adhesion to LECs, but did influence sprout length (FIG. 18 A-B). Previous studies have shown that Gal8 binding to Pdpn favours clustering and activation of integrin beta1 (CD29) in LECs. It was here demonstrated that attachment of PDPN+ BMDMs to LEC sprouts could be blocked by inhibiting integrin beta 1 (integrin B1 or CD29). (FIG. 19A-B). The attraction of PDPN+ BMDMs to soluble galectin-8 could likewise be blocked by inhibiting integrin B1 (FIG. 19C).

To assess the functional relevance of these findings, a capillary network formation assay was performed where Pdpn WT or KO BMDMs were seeded together with LECs on a Matrigel layer. In these conditions LECs cultured with Pdpn KO BMDMs formed shorter sprouts than with WT BMDMs, suggesting that BMDM to LEC adhesion supports lymphatic sprout formation (FIG. 20). Altogether, our data show that lymphatic Gal8 is important for macrophage migration and adhesion to the lymphatic sprouts which in turn will be supported and maintained by these neighbouring macrophages.

### Example 7. Discussion

In sum, by reporting mouse and human data, it is here proven that Podoplanin (a lymphatic lineage marker) defines a macrophage differentiation state/subset that is responsible for lymph vessel maintenance and sprouting in the tumor by their close interaction. This may be on lymph vessel level similar to what has been previously described for Tie2-expressing macrophages (TEMs) in the process of tumor blood vessel formation (De Palma et al. 2017, Nat Rev Cancer 17:457-474). Pdpn+ macrophages were not found in normal skin or in wounded corneas.

It was shown that Pdpn in Pdpn-expressing TAMs is engaging beta1 integrin during the recruitment and adhesion process to lymph vessels where they promote sustained lymphangiogenesis and lymphatic metastasis in breast cancer. Gal8 was confirmed to be a bridging molecule between podoplanin-expressing macrophages and lymphatic vessels in breast cancer.

Unlike VEGF-C that plays an essential role in lymphatic homeostasis as well as physiological and pathological angiogenesis, the pathway described here holds specificity for tumor lymphangiogenesis, therefore circumventing possible side effects (such as lymph oedema) observed in humans and mice when treating the tumor with anti-VEGF-C.

Gal8, integrin beta 1 (on podoplanin-positive macrophages), and podoplanin on podoplanin-positive macrophages are candidate target to prevent tumor metastatis and tumor-induced lymphatic growth. Gal8 expression or Pdpn+ macrophage localization to the lymph vessel can be interrogated in order to predict disease outcome and progression.

### Example 8. Generation of Multispecific Antigen Binding Molecules Specifically Binding to Podoplanin-Positive Macrophages/PEMs and Testing for PEM-Neutralization

As described in Example 1, is not expressed in all tumor-infiltrating immune cells, but exclusively on a subset (about 30%) of the tumor-associated macrophages (TAMs). As described in Examples 2 and 5, selective TAM-specific genetic knockout of podoplanin expression is blocking metastasis and peritumoral lymphangiogenesis. As described in Example 4, the presence of podoplanin expressing TAMs is uniquely associated with lymphatic vessels in human breast cancer patients, therewith confirming mice data (Example 3).

It was thus shown that expression of podoplanin on TAMs is required for the migration and adhesion of TAMs to lymphatic endothelial cells. In mice, macrophage-specific deletion of podoplanin does not affect total TAM infiltration but rather prevents their localization around the lymphatics in several breast cancer models. As a consequence, lymphatic vessels display a functional and density deficit (while blood vessels are not affected). Reduced lymph vessel number and functionality strongly prevents breast cancer metastasis while lymph vessels in other organs do not change. Intratumoral injection of a Gal8 inhibitor mimicked this phenotype in wild-type mice but did not display any effect when TAMs were deficient for podoplanin. In breast cancer patients, podoplanin expressing macrophage association to lymphatics or lymph vessel-born Gal8 expression strongly correlates with the incidence of lymph node metastasis. These findings highlight the functional role of podoplanin in podoplanin expressing macrophages and open new possibilities to specifically target tumor metastasis, tumor-induced lymphangiogenesis by using podoplanin expressing macrophages (and thus not podoplanin expressed in e.g. lymph vessel cells and/or lymph node cells) as a specific target.

One way of specifically targeting podoplanin on podoplanin expressing macrophages is by coupling a podoplanin-specific antigen binding molecule (such as an antibody or any antigen-binding fragment thereof, nanobody, darpin, etc.) with a second antigen binding molecule wherein the latter is binding to a surface marker different from podoplanin but likewise preferentially expressed by podoplanin expressing macrophages. Example 1 herein identified CD206 (macrophage mannose receptor), CD86, and CD204 as initial candidates of surface markers specific for podoplanin expressing macrophages (compared to tumor-associated macrophages not expressing podoplanin).

Thus, a camelid (such as llama) is immunized with podoplanin and podoplanin-binding camelid antibodies, as well as their podoplanin-binding VHH fragments, are isolated and characterized.

Camelid VHH fragments binding to CD206 are known in the art (e.g. WO 2013/174537—CDR1 sequences therein: SEQ ID Nos 67-96; CDR2 sequences therein: SEQ ID Nos:127-156; CDR3 sequences therein: SEQ ID Nos:187-216; WO 2017/158436—nanobody sequences therein: SEQ ID NOs: 30-56, 58, 60, 62, 64, 66, 68, 70, 72, 74, 76, 78, 80, 82, 84, 86, 88, 90, 92, 94, 96, 98, 100, 102, 104, 106, 108, and 112). A CD206-binding VHH fragment is, by means of a flexible linker (such as (GlyₙSer)ₘ or (GlyGly)ₙ linker (described supra), coupled to a podoplanin-binding VHH fragment. The resulting bispecific antigen binding compound is subsequently produced in E. coli and purified. After purification, binding specificity of the bispecific antigen binding compound is first confirmed in vitro by showing preferential binding to podoplanin expressing macrophages (such as isolated from the tumor environment, such as a breast tumor environment) compared to macrophages not expressing podoplanin, which immediately reveals applicability in diagnostic methods/kits. Subsequently the effect of the podoplanin/CD206-bispecific antigen binding compound is assessed in vivo similarly as described in Example 6 (substituting TDG for the podoplanin/CD206-bispecific antigen binding compound), which reveals therapeutic applicability.

Independent immunization campaigns are set up to obtain camelid antibodies binding CD86 and CD204. Similarly as described above, podoplanin/CD86-bispecific antigen binding compounds and podoplanin/CD204-bispecific antigen binding compounds are prepared and assessed as described above.

1. A method for determining metastasis status of a tumor, comprising the steps of:
obtaining from a mammal having the tumor a sample comprising lymphatic vessel cells and/or lymph node cells;
determining presence or absence of podoplanin expressing macrophages (PEMs) in said sample;
determining the tumor to be metastatic when PEMs are present in said sample.

2. The method according to claim 1, wherein said lymphatic vessel cells and/or lymph node cells are peritumoral lymphatic vessel cells and/or lymph node cells.

3. The method according to claim 1, wherein the obtained sample is processed for detection of lymph vessel cells and/or lymph node cells, and wherein the sample is processed for detection of PEMs.

4. The method according to claim 3, wherein said processing comprises detection of podoplanin expressed on the lymph vessel cells and/or lymph node cells and of podoplanin expressed on PEMs.

5. The method according to claim 4, wherein said processing further comprises detection of a macrophage marker different from podoplanin.

6. A method for determining surface markers specific to PEMs, comprising the steps of:
obtaining a macrophage population expressing podoplanin and a macrophage population not expressing podoplanin;
reacting the obtained macrophage populations with a reagent specific to at least one surface molecule suspected to be present on macrophages of at least one population, wherein said surface molecule is different from podoplanin; and
characterizing a surface molecule for which the ratio of its levels present on PEMs over its levels present on macrophages not expressing podoplanin is higher than 1 as surface marker specific to PEMs.

7. The method according to claim 6 wherein the detected surface molecule specific for PEMs and different from podoplanin is the macrophage mannose receptor CD206, is CD86, or is CD204.

8. A method of screening for compounds neutralizing PEMs, comprising the steps of:
obtaining PEMs and lymphatic vessel cells and/or with lymph node cells;
assessing the interaction of the PEMs with lymphatic vessel cells and/or with lymph node cells in the presence or absence of at least one compound candidate for inhibiting such interaction;
selecting a compound capable of inhibiting interaction of the PEMs with the lymphatic vessel cells and/or with the lymph node cells as compound capable of neutralizing PEMs; and, optionally,
combining said selected compound with at least one pharmaceutically excipient to produce a pharmaceutical composition.

9. A method of screening for compounds neutralizing PEMs, comprising the steps of:
obtaining PEMs and galectin-8;
assessing the interaction of the PEMs with galectin-8 in the presence or absence of at least one compound candidate for inhibiting such interaction;
selecting a compound capable of inhibiting interaction between the PEMs and galectin-8 as compound capable of neutralizing PEMs; and, optionally,
combining said selected compound with at least one pharmaceutically excipient to produce a pharmaceutical composition.

10. A method of screening for compounds binding specifically to PEMs, wherein the compound is not binding to podoplanin on PEMs, and wherein the method is comprising the steps of:
obtaining a macrophage population expressing podoplanin and a macrophage population not expressing podoplanin;
reacting the macrophage populations with at least one compound; and
selecting a compound for which the ratio of its level of binding on PEMs over its levels of binding on macrophages not expressing podoplanin is higher than 1, and which is not binding to podoplanin on PEMs, as compound binding specifically to PEMs.

11. The method according to any of claims 6 to 10 wherein said PEMs or macrophages expressing podoplanin are obtained from a tumor, a tumor environment and/or from a tumor stroma.

12. A multi-specific antigen binding molecule specifically binding to PEMs, wherein said molecule is at least binding to podoplanin and to a second PEM-specific surface molecule.

13. The multi-specific antigen binding molecule according to claim 12 wherein the second PEM-specific surface molecule is chosen from the macrophage mannose receptor CD206, CD86, and CD204.

14. The multi-specific antigen binding molecule according to claim 12 or 13 further comprising a detectable label.

15. A kit for determining metastasis status of a tumor comprising at least one agent for detection of PEMs.

16. The kit according to claim 15 further comprising at least one agent for detection of lymph vessel cells and/or lymph node cells.

17. The kit according to claim 15 or 16 wherein the at least one agent for detection of PEMs is the multi-specific antigen binding molecule according to any of claims 12 to 14.

18. The method according to any of claims 1 to 5 wherein said PEMs are detected by means of a multi-specific antigen binding molecule according to any of claims 12 to 14.

19. A compound neutralizing PEMs for use in treating or inhibiting lymphatic metastasis of a primary tumor or for use in treating or inhibiting tumor-induced lymphangiogenesis.

20. A compound neutralizing PEMs for use in treating or inhibiting lymphatic metastasis of a primary tumor, wherein the metastasis status of a tumor is determined with a method according to any of claims 1 to 5.

21. The compound neutralizing PEMs for use according to claim 12 or 13 wherein the compound is neutralizing interaction between PEMs and a lymphatic vessel, a lymphatic vessel cell, a lymph node, or a lymph node cell.

22. The compound neutralizing PEMs for use according to claim 2 or 12 which is an antibody binding to podoplanin, galectin-8 or integrin beta 1, or is an antigen-binding fragment thereof.

23. A method, compound or kit according to any of the preceding claims wherein said tumor is a breast tumor or is breast cancer.
